(19) United States
(12) Patent Application Publication
(10) Pub. No.: US 2023/0143099 A1
(43) Pub. Date: May 11, 2023

(54) BREATHING RHYTHM RESTORATION SYSTEMS, APPARATUSES, AND INTERFACES AND METHODS FOR MAKING AND USING SAME

(71) Applicant: Quantum Interface,LLC, Austin, TX (US)

(72) Inventors: Naomi Josephson, Austin, TX (US); Jonathan Josephson, Austin, TX (US)

(73) Assignee: QUANTUM INTERFACE, LLC, Austin, TX (US)

(21) Appl. No.: 17/887,473

(22) Filed: Aug. 14, 2022

Related U.S. Application Data
(60) Provisional application No. 63/233,240, filed on Aug. 14, 2021.

(51) Int. Cl. G09B 23/28; A61B 5/087; G09B 9/00

(52) U.S. Cl. CPC G09B 23/28; A61B 5/087; G09B 9/00

(57) ABSTRACT

Apparatuses, systems, and interfaces and methods implementing them, wherein apparatuses, systems, and interfaces are configured to assist or aide a user experiencing an adverse breathing event to reestablish a normal or healthy breathing rhythm or pattern quickly and efficiently via simulated breathing rhythms or patterns including audio recordings or simulations, visual recordings or simulations, audiovisual recordings or simulations, or avatar recordings or simulations.

### RELATED APPLICATIONS

This application claims the benefit of and priority to U.S. Provisional Patent Application Ser. No. 63/233,240 filed 14 Aug. 2022.

United States Patent Published Application Nos. 20170139556 published May 18, 2017, 20190391729 published Dec. 26, 2019, WO2018237172 published Dec. 27, 2018, WO2021021328 published Feb. 4, 2021, and U.S. Pat. No. 7,831,932 issued Nov. 9, 2010, U.S. Pat. No. 7,861,188 issued Dec. 28, 2010, U.S. Pat. No. 8,788,966 issued Jul. 22, 2014, U.S. Pat. No. 9,746,935 issued Aug. 29, 2017, U.S. Pat. No. 9,703,388 issued Jul. 11, 2017, U.S. Pat. No. 11,256,337 issued Feb. 22, 2022, U.S. Pat. No. 10,289,204 issued May 14, 2019, U.S. Pat. No. 10,503,359 issued Dec. 10, 2019, U.S. Pat. No. 10,901,578 issued Jan. 26, 2021, U.S. Pat. No. 11,221,739 issued Jan. 11, 2022, U.S. Pat. No. 10,263,967 issued Apr. 16, 2019, U.S. Pat. No. 10,628,977 issued Apr. 21, 2020, U.S. Pat. No. 11,205,075 issued Dec. 21, 2021, U.S. Pat. No. 10,788,948 issued Sep. 29, 2020, and U.S. Pat. No. 11,226,714 issued Jan. 18, 2022, are incorporated by reference via the application of the Closing Paragraph.

### BACKGROUND OF THE DISCLOSURE

### 1. Field of the Disclosure

Embodiments of the present disclosure relate to apparatuses, systems, and interfaces and methods implementing them to assist or aide a user experiencing an adverse breathing event to reestablish a normal breathing rhythm or pattern quickly and efficiently via a generated visual, audio, audiovisual, and/or haptic breathing rhythm or pattern.

In particular, embodiments of the present disclosure relate to apparatuses, systems, and interfaces and methods implementing them, wherein the apparatuses, systems, or interfaces and methods implementing them are designed to simulate a visual, audio, audiovisual, and/or haptic breathing rhythm or pattern to assist or aide a user experiencing an adverse breathing event to reestablish a normal breathing rhythm or pattern quickly and efficiently, wherein the simulated visual pattern changes in accord with the user's normal breathing pattern, simulated audio pattern changes in accord with the user's normal breathing pattern, simulated audiovisual pattern changes in accord with the user's normal breathing pattern, and/or simulated haptic pattern changes in accord with the user's normal breathing pattern, and wherein the simulated patterns may also show a difference between the user's current breathing patterns and the user's normal breathing patterns to further assist or aide the user to reestablish a normal breathing pattern as the user experiencing an adverse breathing event and may highlight the differences visually, acoustically, haptically, or any combination thereof.

### 2. Description of the Related Art

While there are numerous systems and methods for monitoring and outputting simulations to assist a user in restoring a health breathing rhythm/pattern, especially for users suffering from asthmatics, allergies, anxieties, atypical breathing rhythms/patterns, or any malady that causes breathing abnormalities, there is still a need in the art for new and novel systems and methods for simulating breathing rhythms/patterns during an adverse breathing event to assist a user in restoring a health breathing rhythm/pattern.

### SUMMARY OF THE DISCLOSURE

Embodiments of this disclosure provide breathing rhythm/pattern apparatuses, systems, or interfaces, wherein the apparatuses, systems, and/or interfaces include a processing unit having memory, communications hardware and software, one or more mass storage devices, and a power supply coupled to or associated with one or more input devices and one or more output devices and wherein the apparatuses, systems, and/or interfaces are configured to acquire or capture user breathing data during an adverse breathing event and output audio recorded breathing patterns or rhythms, visual recorded breathing patterns or rhythms, audiovisual recorded breathing patterns or rhythms, and/or simulated/computer generated breathing patterns or rhythms to help the user recover from the adverse breathing event. In certain embodiments, the apparatuses, systems, and/or interfaces are configured to: (a) activate the apparatuses, systems, or interfaces (1) via the apparatuses, systems, and/or interfaces detecting an adverse breathing event or (2) via a user input; (b) acquire, capture, and/or receive initial breathing data from the user undergoing an adverse breathing event, (c) select a breathing output comprising audio recorded breathing pattern or rhythm, visual recorded breathing pattern or rhythm, audiovisual recorded breathing pattern or rhythm, and/or simulated/computer generated breathing pattern or rhythm (1) based on input from the user or (2) based on an automatic selection by the apparatuses, systems, and/or interfaces; (d) continue to acquire, capture, and/or receive breathing data from the user, (e) monitor the user breathing data, and (f) modify the breathing output based on the acquired breathing data. In other embodiments, the breathing output is selected to best assist the user in reestablishing a normal breathing pattern or rhythms based on the nature of the adverse breathing event the user is experiencing.

Embodiments of this disclosure provide methods for implementing the breathing rhythm/pattern apparatuses, systems, and/or interfaces, wherein the apparatuses, systems, and/or interfaces include a processing unit having memory, communications hardware and software, one or more mass storage devices, and a power supply coupled to or associated with one or more input devices and one or more output devices and wherein the methods include acquiring or capturing user breathing data during an adverse breathing event and outputting audio recorded breathing patterns or rhythms, visual recorded breathing patterns or rhythms, audiovisual recorded breathing patterns or rhythms, and/or simulated/computer generated (CG) breathing patterns or rhythms to help the user recover from the adverse breathing event. In certain embodiments, the methods include: (a) activating the apparatuses, systems, and/or interfaces either (1) via the apparatuses, systems, and/or interfaces detecting an adverse breathing event or (2) via an input from the user; (b) acquiring, capturing, and/or receiving initial breathing data from the user undergoing an adverse breathing event, (c) selecting a breathing output comprising audio recorded breathing pattern or rhythm, visual recorded breathing pattern or rhythm, audiovisual recorded breathing pattern or rhythm, and/or simulated/computer generated breathing pattern or rhythm (1) based on input from the user or (2) based on an automatic selection by the apparatuses, systems, and/or interfaces; (d) continuing acquiring, capturing, and/or receiving breathing data from the user, (e) monitoring the user breathing data, and (f) modifying the breathing output based on the acquired, captured, and/or received breathing data. In other embodiments, the breathing output is selected to best assist the user in reestablishing a normal breathing pattern or rhythms based on the nature of the adverse breathing event the user is experiencing.

### BRIEF DESCRIPTION OF THE DRAWINGS OF THE DISCLOSURE

The disclosure may be better understood with reference to the following detailed description together with the appended illustrative drawings in which like elements are numbered the same:

FIG. 1A depicts several breathing rhythms, Rhythms 1-4, which are different known standard breathing rhythms or patterns.

FIG. 1B depicts Rhythms 1-4 with stepwise audio recordings or simulations.

FIG. 1C depicts Rhythms 1-4 with continuous audio recordings or simulations.

FIG. 1D depicts Rhythms 1-4 with grey scale visual recordings or simulations.

FIG. 1E depicts Rhythms 1-4 with color scale visual recordings or simulations, wherein the color may be any color and the scale comprising shades of the color.

FIG. 1F depicts Rhythms 1-4 with stepwise audio recordings or simulations and grey scale visual recordings or simulations.

FIG. 1G depicts Rhythms 1-4 with continuous audio recordings or simulations and grey scale visual recordings or simulations.

FIG. 1H depicts Rhythms 1-4 with stepwise audio recordings or simulations and color scale visual recordings or simulations, wherein the color may be any color and the scale comprising shades of the color.

FIG. 1I depicts Rhythms 1-4 with continuous audio recordings or simulations and color scale visual recordings or simulations, wherein the color may be any color and the scale comprising shades of the color.

FIG. 2A-I depict different continuation audio recording or simulation formats some including volume increases and volume pauses or volume decreases and volume pauses.

FIG. 3A-H depict different visual recordings and simulations involving grey scale circles in different arrangements.

FIG. 3I-J depict different visual recordings and simulations involving circles that increase during inhalations or decrease during exhalations.

FIG. 3K-L depict different visual recordings and simulations involving grey scale circles that increase during inhalations or decrease during exhalations.

FIG. 3M-N depict different visual recordings and simulations involving squares that increase during inhalations or decrease during exhalations.

FIG. 3O-P depict different visual recordings and simulations involving color scale squares that increase during inhalations or decrease during exhalations, wherein the color may be any color and the scale comprising shades of the color.

FIG. 4A depicts an inhalation and exhalation simulation including stepwise audio recordings or simulations and a stepwise sunset visualization that grows in intensity during inhalation and fades in intensity during exhalation.

FIG. 4B depicts an inhalation and exhalation simulation including continuous audio recordings or simulations and a stepwise the sunset visualization that grows in intensity during inhalation and fades in intensity during exhalation.

FIG. 5A depicts an inhalation and exhalation simulation including stepwise audio recordings or simulations and a stepwise sunrise visualization that grows in intensity during inhalation and fades in intensity during exhalation.

FIG. 5B depicts an inhalation and exhalation simulation including continuous audio recordings or simulations and a stepwise the sunrise visualization that grows in intensity during inhalation and fades in intensity during exhalation.

FIG. 6A depicts an inhalation and exhalation simulation including stepwise audio recordings or simulations and a stepwise spiral galaxy visualization that grows in intensity during inhalation and fades in intensity during exhalation.

FIG. 6B depicts an inhalation and exhalation simulation including continuous audio recordings or simulations and a stepwise the spiral galaxy visualization that grows in intensity during inhalation and fades in intensity during exhalation.

FIG. 7A depicts an inhalation and exhalation simulation including stepwise audio recordings or simulations and a stepwise Milky Way galaxy visualization that grows in intensity during inhalation and fades in intensity during exhalation.

FIG. 7B depicts an inhalation and exhalation simulation including continuous audio recordings or simulations and a stepwise the Milky Way galaxy visualization that grows in intensity during inhalation and fades in intensity during exhalation.

FIG. 8A depicts an inhalation and exhalation simulation including stepwise audio recordings or simulations and a stepwise Andromeda galaxy visualization that grows in intensity during inhalation and fades in intensity during exhalation.

FIG. 8B depicts an inhalation and exhalation simulation including continuous audio recordings or simulations and a stepwise the Andromeda galaxy visualization that grows in intensity during inhalation and fades in intensity during exhalation.

FIG. 9A depicts an inhalation and exhalation simulation including stepwise audio recordings or simulations and a stepwise Hubble telescope star event image that grows in intensity during inhalation and fades in intensity during exhalation.

FIG. 9B depicts an inhalation and exhalation simulation including continuous audio recordings or simulations and a stepwise the Hubble telescope star event image that grows in intensity during inhalation and fades in intensity during exhalation.

FIG. 10A depicts an inhalation and exhalation simulation including stepwise audio recordings or simulations and a stepwise moon set image that grows in intensity during inhalation and fades in intensity during exhalation.

FIG. 10B depicts an inhalation and exhalation simulation including continuous audio recordings or simulations and a stepwise the moon set image that grows in intensity during inhalation and fades in intensity during exhalation.

FIGS. 11A-AI depicts a sequence of simulated avatar images that show activating the avatar and displaying the avatar using a translucent ball to simulate breathing (ball gets bigger during inhalation and smaller during exhalation) and text box displaying text messages to the user as breathing resumes to normal and the information in the text boxes may be heard acoustically or haptically through pulse technology.

### DEFINITIONS USED IN THE DISCLOSURE

The term “at least one”, “one or more”, and “one or a plurality” mean one thing or more than one thing with no limit on the exact number; these three terms may be used interchangeably within this application. For example, at least one device means one or more devices or one device and a plurality of devices.

The term “about” means that a value of a given quantity is within ±20% of the stated value. In other embodiments, the value is within ±15% of the stated value. In other embodiments, the value is within ±10% of the stated value. In other embodiments, the value is within ±7.5% of the stated value. In other embodiments, the value is within ±5% of the stated value. In other embodiments, the value is within ±2.5% of the stated value. In other embodiments, the value is within ±1% of the stated value.

The term “substantially” or “essentially” means that a value of a given quantity is within ±10% of the stated value. In other embodiments, the value is within ±7.5% of the stated value. In other embodiments, the value is within ±5% of the stated value. In other embodiments, the value is within ±2.5% of the stated value. In other embodiments, the value is within ±1% of the stated value. In other embodiments, the value is within ±0.5% of the stated value. In other embodiments, the value is within ±0.1% of the stated value.

The term “hard select” or “hard select protocol” or “hard selection” or “hard selection protocol” means a mouse click or double click (right and/or left), keyboard key strike, tough down event, lift off event, touch screen tab, haptic device touch, voice command, hover event, eye gaze event, or any other action that required a user action to generate a specific output to affect a selection of an object or item displayed on a display device. The term “voice command” means an audio command sensed by an audio sensor. The term “neural command” means a command sensed by a sensor capable of reading neurological states—mind control.

The term “motion” and “movement” are often used interchangeably and mean motion or movement that is capable of being detected by a motion sensor within an active zone of the sensor, wherein the motion may have properties including direction, speed, velocity, acceleration, magnitude of acceleration, and/or changes of any of these properties over a period of time. Thus, if the sensor is a forward viewing sensor and is capable of sensing motion within a forward extending conical active zone, then movement of anything within that active zone that meets certain threshold detection criteria, will result in a motion sensor output, where the output may include at least direction, angle, distance/displacement, duration (time), velocity, and/or acceleration. Moreover, if the sensor is a touch screen or multi-touch screen sensor and is capable of sensing motion on its sensing surface, then movement of anything on that active zone that meets certain threshold detection criteria, will result in a motion sensor output, where the output may include at least direction, angle, distance/displacement, duration (time), velocity, and/or acceleration. Of course, the sensors do not need to have threshold detection criteria, but may simply generate output anytime motion or any kind is detected. The processing units can then determine whether the motion is an actionable motion or movement and a non-actionable motion or movement.

The term “motion sensor” or “motion sensing component” means any sensor or component capable of sensing motion of any kind by anything with an active zone—area or volume, regardless of whether the sensor's or component's primary function is motion sensing. Of course, the same is true of sensor arrays regardless of the types of sensors in the arrays or for any combination of sensors and sensor arrays.

The term “gaze controls” means taking gaze tracking input from sensors and converting the output into control features including all type of commands. The sensors may be eye and/or head tracking sensors, where the sensor may be processors that are in communication with mobile or non-mobile apparatuses including processors. In VR/AR/MR/XR applications using mobile or non-mobile devices, the apparatuses, systems, and interfaces of this disclosure may be controlled by input from gaze tracking sensors, from processing gaze information from sensors on the mobile devices or non-mobile devices or communication with the mobile devices or non-mobile devices that are capable of determine gaze and/or posture information, or mixtures and combinations.

The term “eye tracking sensor” means any sensor capable of tracking eye movement such as eye tracking glasses, eye tracking cameras, or any other eye tracking sensor.

The term “head tracking sensor” means any sensor capable of tracking head movement such as head tracking helmets, eye tracking glasses, head tracking cameras, or any other head tracking sensor.

The term “face tracking sensor” means any sensor capable of tracking face movement such as any facial head tracking gear, face tracking cameras, or any other face tracking sensor.

The term “gaze” or “pose” or “pause” means any type of fixed motion over a period of time that may be used to cause an action to occur. Thus, in eye tracking, a gaze is a fixed stare of the eyes or eye over a period of time greater than a threshold, in body, body part, or face tracking, a pose is a stop in movement of the body or body part or holding a specific body posture or body part configuration for a period of time greater than a threshold, and a pause is a stop in motion for a period of time greater than a threshold, that may be used by the systems, apparatuses, interfaces, and/or implementing methods to cause an action to occur.

The term “real object” or “real world object” means real world device, attribute, or article that is capable of being controlled by a processing unit. Real objects include objects or articles that have real world presence including physical, mechanical, electro-mechanical, magnetic, electro-magnetic, electrical, or electronic devices, waveform devices, or any other real world device that may be controlled by a processing unit.

The term “virtual object” means any construct generated in or attribute associated with a virtual world or by a computer and may be displayed by a display device and that are capable of being controlled by a processing unit. Virtual objects include objects that have no real world presence, but are still controllable by a processing unit or output from a processing unit(s). These objects include elements within a software system, product or program such as icons, list elements, menu elements, applications, files, folders, archives, generated graphic objects, 1D, 2D, 3D, and/or nD graphic images or objects, generated real world objects such as generated people, generated animals, generated devices, generated plants, generated landscapes and landscape objects, generate seascapes and seascape objects, generated skyscapes or skyscape objects, 1D, 2D, 3D, and/or nD zones, 2D, 3D, and/or nD areas, 1D, 2D, 3D, and/or nD groups of zones, 2D, 3D, and/or nD groups or areas, volumes, attributes or characteristics such as quantity, shape, zonal, field, affecting influence changes or the like, or any other generated real world or imaginary objects or attributes. Augmented and/or mixed reality is a combination of real and virtual objects and attributes.

The term “entity” means a human or an animal or robot or robotic system (autonomous or non-autonomous or virtual representation of a real or imaginary entity.

The term “entity object” means a human or a part of a human (fingers, hands, toes, feet, arms, legs, eyes, head, body, etc.), an animal or a part of an animal (fingers, hands, toes, feet, arms, legs, eyes, head, body, etc.), or a real world object under the control of a human or an animal or a robot and include such articles as pointers, sticks, or any other real world object that can be directly or indirectly controlled by a human or animal or a robot. In VR/AR environments, the entity object may also include virtual objects.

The term “mixtures” means different objects, attributes, data, data types or any other feature that may be mixed together or controlled together.

The term “combinations” means different objects, attributes, data, data types or any other feature that may be packages or bundled together but remain separate.

The term “sensor data” means data derived from at least one sensor including user data, motion data, environment data, temporal data, contextual data, historical data, waveform data, other types of data, and/or mixtures and combinations thereof.

The term “user data” means user attributes, attributes of entities under the control of the user, attributes of members under the control of the user, information or contextual information associated with the user, or mixtures and combinations thereof.

The terms “user features”, “entity features”, and “member features” means features including: (a) overall user, entity, or member shape, texture, proportions, information, matter, energy, state, layer, size, surface, zone, area, any other overall feature, attribute or characteristic, and/or mixtures or combinations thereof (b) specific user, entity, or member part shape, texture, proportions, characteristics, any other part feature, and/or mixtures or combinations thereof (c) particular user, entity, or member dynamic shape, texture, proportions, characteristics, any other part feature, and/or mixtures or combinations thereof and (d) mixtures or combinations thereof. For certain software programs, routines, and/or elements, features may represent the manner in which the program, routine, and/or element interact with other software programs, routines, and/or elements operate or are controlled. All such features may be controlled, manipulated, and/or adjusted by the motion-based systems, apparatuses, and/or interfaces of this disclosure.

The term “motion data” or “movement data” means data generated by one or more motion sensor or one or more sensors of any type capable of sensing motion/movement comprising one or a plurality of motions/movements detectable by the motion sensors or sensing devices.

The term “motion properties” or “movement properties” means properties associated with the motion data including motion/movement direction (linear, curvilinear, circular, elliptical, etc.), motion/movement distance/displacement, motion/movement duration (time), motion/movement velocity (linear, angular, etc.), motion/movement acceleration (linear, angular, etc.), motion signature or profile—manner of motion/movement (motion/movement properties associated with the user, users, objects, areas, zones, or combinations of thereof), dynamic motion properties such as motion in a given situation, motion learned by the system based on user interaction with the systems, motion characteristics based on the dynamics of the environment, influences or affectations, changes in any of these attributes, and/or mixtures or combinations thereof. Motion or movement based data is not restricted to the movement of a single body, body part, and/or member under the control of an entity, but may include movement of one or any combination of movements of any entity and/or entity object. Additionally, the actual body, body part and/or member's identity is also considered a movement attribute. Thus, the systems/apparatuses, and/or interfaces of this disclosure may use the identity of the body, body part and/or member to select between different set of objects that have been pre-defined or determined based on environment, context, and/or temporal data.

The term “gesture” or“predetermine movement pattern” means a predefined movement or posture preformed in a particular manner such as closing a fist lifting a finger that is captured compared to a set of predefined movements that are tied via a lookup table to a single function and if and only if, the movement is one of the predefined movements does a gesture based system actually go to the lookup and invoke the predefined function.

The term “environment data” means data associated with the user's surrounding or environment such as location (GPS, etc.), type of location (home, office, store, highway, road, etc.), extent of the location, context, frequency of use or reference, attributes, characteristics, and/or mixtures or combinations thereof.

The term “temporal data” means data associated with duration of motion/movement, events, actions, interactions, etc., time of day, day of month, month of year, any other temporal data, and/or mixtures or combinations thereof.

The term “historical data” means data associated with past events and characteristics of the user, the objects, the environment and the context gathered or collected by the systems over time, or any combinations of these.

The term “contextual data” means data associated with user activities, environment activities, environmental states, frequency of use or association, orientation of objects, devices or users, association with other devices and systems, temporal activities, any other content or contextual data, and/or mixtures or combinations thereof.

The term “predictive data” means any data from any source that permits that apparatuses, systems, interfaces, and/or implementing methods to use data to modify, alter, change, augment, update, enhance, reformat, restructure, and/or redesign a virtual training routine, exercise, program, etc. to better tailor the training routine, exercise, program, etc. for each user or for all users, where the changes may be implemented before, during and after a training session.

The term “simultaneous” or “simultaneously” means that an action occurs either at the same time or within a small period of time. Thus, a sequence of events are considered to be simultaneous if they occur concurrently or at the same time or occur in rapid succession over a short period of time, where the short period of time ranges from about 1 nanosecond to 5 second. In other embodiments, the period ranges from about 1 nanosecond to 1 second. In other embodiments, the period ranges from about 1 nanosecond to 0.5 seconds. In other embodiments, the period ranges from about 1 nanosecond to 0.1 seconds. In other embodiments, the period ranges from about 1 nanosecond to 1 millisecond. In other embodiments, the period ranges from about 1 nanosecond to 1 microsecond. It should be recognized that any value of time between any stated range is also covered.

The term “and/or” means mixtures or combinations thereof so that whether an “and/or” connectors is used, the “and/or” in the phrase or clause or sentence may end with “and mixtures or combinations thereof”.

The term “spaced apart” means for example that objects displayed in a window of a display device are separated one from another in a manner that improves an ability for the systems, apparatuses, and/or interfaces to discriminate between objects based on movement sensed by motion sensors associated with the systems, apparatuses, and/or interfaces.

The term “maximally spaced apart” means that objects displayed in a window of a display device are separated one from another in a manner that maximizes a separation between the objects to improve an ability for the systems, apparatuses, and/or interfaces to discriminate between objects based on motion/movement sensed by motion sensors associated with the systems, apparatuses, and/or interfaces.

The term “s” means one or more seconds. The term “ms” means one or more milliseconds (10⁻³ seconds). The terms “μs” means one or more micro seconds (10⁻⁶ seconds). The term “ns” means nanosecond (10⁻⁹ seconds). The term “ps” means pico second (10⁻¹² seconds). The term “fs” means femto second (1(1⁻¹⁵ seconds). The term “as” means femto second (10⁻¹⁸ seconds).

The term “hold” means to remain stationary at a display location for a finite duration generally between about 1 ms to about 2 s.

The term “brief hold” means to remain stationary at a display location for a finite duration generally between about 1 μs to about 1 s.

The term “microhold” or “micro duration hold” means to remain stationary at a display location for a finite duration generally between about 1 as to about 500 ms. In certain embodiments, the microhold is between about 1 fs to about 500 ms. In certain embodiments, the microhold is between about 1 ps to about 500 ms. In certain embodiments, the microhold is between about 1 ns to about 500 ms. In certain embodiments, the microhold is between about 1 μs to about 500 ms. In certain embodiments, the microhold is between about 1 ms to about 500 ms. In certain embodiments, the microhold is between about 100 us to about 500 ms. In certain embodiments, the microhold is between about 10 ms to about 500 ms. In certain embodiments, the microhold is between about 10 ms to about 250 ms. In certain embodiments, the microhold is between about 10 ms to about 100 ms.

The term “VR” means virtual reality encompassing two-dimension (2D), three-dimension (3D), four-dimensional (4D), or multi-dimensional (nD) computer-generated environments that include computer-generated (CG) two-dimension (2D), three-dimension (3D), four-dimension (4D), and/or multi-dimensional (nD) (a) made up or imaginary objects, items, constructs, images, scenes, and/or environments, (b) GC simulated real world objects, items, images, scenes, and/or environments, and/or (c) attributes associated therewith, wherein some or all of the objects, items, constructs, images, scenes, environments and/or attributes may be interacted with by a user. In certain embodiments, the computer-generated objects, items, images, scenes, environments, and/or attributes associated therewith may be interacted by a user equip with specialized electronic equipment, such as eye tracking glasses, head and eye helmets, VR visors, gloves equip with sensors, and/or body suits equip with sensors.

The term “AR” means augmented reality, which is a technology that superimposes computer-generated objects, items, images, scenes, environments, and/or attributes associated therewith on a real world environment, wherein some or all of the objects, items, constructs, images, scenes, environments and/or attributes may be interacted with by a user.

The term “MR” means mixed reality is a blend (a) made up or imaginary objects, items, constructs, images, scenes, environments, and/or attributes associated therewith and (b) GC simulated real world objects, items, images, scenes, environments, and/or attributes associated therewith. The two worlds are “mixed” together to create a realistic environment. A user may navigate this environment and interact with both real and virtual objects, items, images, scenes, environments, and/or attributes. Mixed reality (MR) combines aspects of virtual reality (VR) and augmented reality (AR). It sometimes called “enhanced” AR since it is similar to AR technology, but provides more physical interaction.

The term “XR” means extended reality and refers to all real-and-virtual combined environments and human-machine interactions generated by computer technology and wearables. The levels of virtuality range from partially sensory inputs to immersion virtuality, also called VR.

The terms VR is generally used to mean environments that are totally computer generated, while AR, MR, and XR are sometimes used interchangeable to mean any environment that includes real content and virtual or computer generated content. We will often use the AR/MR/XR as a general term for all environments that including real content and virtual or computer generated content, and these terms may be used interchangeably.

### DETAILED DESCRIPTION OF THE DISCLOSURE

The inventors have found that normal breathing patterns or rhythms may be restored by producing a computer simulated output comprising an audiovisual sequence including visual and audio that rises and falls with of a wave motion, an audiovisual sequence of wind motion, an audiovisual sequence of sun rising and sitting motion, an audiovisual sequence of moon rising and setting motion, an audiovisual sequence of star rising and sitting motion, an audiovisual sequence of plants growing and flowering, an audiovisual sequence of animal sequence motion, an audiovisual sequence of computer generated natural, virtual motion, or mixed motion, or mixtures thereof designed to restore a normal breathing pattern.

The inventors have found that breathing exercises may be generated to help people to learn how to breathe, and practice breathing, in a certain fashion to help with calming themselves and to help with wellness. Currently, there are many breathing techniques, and most are taught by counting “inhale 1-2-3-4, hold, exhale 1-2-3-4, hold . . . ” etc. There is also a use of the words “SO (breathe in) . . . Ham (breathe out)” as someone says these words with the desired tempo for breathing rate. The obnoxious part of all this is that you always hear counting, even when you do this on your own, or the use of a meditation word, which is part of a Buddhist or Hindu faith, and many do not want to use these.

The inventors have found that using sounds of nature, such as waves, where the wave sounds are timed so as a person breathes in with the sound of the wave coming into shore, and exhales with the sound of waves retreating. These sounds may be digitally manipulated to match the breathing tempo needs of a person, such as starting with a 4-1-4-1 pattern, then slow down to a 4-1-6-1 pattern as the user relaxes. The apparatuses and systems and interfaces or method implementing the apparatuses or systems may also include one or more virtual elements, such as an AR or VR displayed avatar, that expands and contracts it's torso to the tempo of the audio, or just a glowing ball, or anything dynamic. The apparatuses and systems and interfaces or method implementing the apparatuses or systems may also chimes or other cues of when to start the inhale and exhale. The big difference is that the apparatuses and systems and/or interfaces or method implementing the apparatuses or systems use the flow of the waves, wind, rain, etc., which are timed in accord with the user normal breathing pattern or rhythm to help or aide guidance to the user to reestablish the user's normal breathing pattern or rhythm. For example, the wave builds as it comes into shore and crests, then spreads across the sand, then begins to retreat, picks up speed, has turbulence, and then has a brief rest before the next wave comes in, in accord with the user's normal breathing pattern or rhythm, e.g., the wave pattern to matches user's normal breathing pattern or rhythm.

For example, the apparatuses, systems, and/or interfaces may be configured to manipulate timing of waves by Mammary (our music contractor) and via guitar playing or the playing of any other instrument or collection or assembly of instruments. In certain embodiments, there is no steady tempo for the waves or guitar (any other audio output) in between the inhale/exhale starting points—the music increases and ebbs just like the energy in a wave.

An example of how music may be created to line up with the wave form, so the first notes are slower, then build in volume and the amounts of notes increase mimicking the true “wave's form”, then slows down and amplitude decreases. This virtual wave, made with notes, can also be digitally manipulated with software so any breathing pattern can be guided by the audio (or visual, or both). It's all about synchronizing the breath and turbulence in the lungs with nature.

In certain embodiments, apparatuses, systems, and/or interfaces is configured to modify the audio output to represent the air turbulence in the lungs, which lines up with the same type of turbulence from waves, wind (anything following fluid or field dynamics), and the use of sounds to mimic the turbulence of waves, breathing, air, wind, rain, etc. to guide the user into the right pace of breathing or to help the user reestablish a normal breathing pattern or rhythm after experiencing an adverse breathing event.

The inventors breathing methodology may be use with massage therapy, so that the inhale and sound of the waves coming in lines up with each massage stroke moving towards the heart, and each exhale lines up with moves away from the heart.

The apparatuses or systems and/or interfaces and/or methods implementing the apparatuses or systems are configured to acquire, capture, receive, and/or record normal breathing audio, visual, audiovisual, and/or haptic data of a user experiencing normal breathing and generating user normal breathing audio, visual, audiovisual, and/or haptic recordings or simulated user normal breathing audio, visual, audiovisual, and/or haptic pattern or rhythm from the normal breathing audio, visual, audiovisual, and/or haptic data.

The apparatuses or systems and/or interfaces and/or methods implementing the apparatuses or systems are configured to output a normal breathing recording or a normal breathing simulated pattern or rhythm.

Embodiments of this disclosure broadly relate to breathing rhythm/pattern apparatuses, systems, or interfaces, wherein the apparatuses, systems, and/or interfaces include a processing unit having memory, communications hardware and software, one or more mass storage devices, and a power supply coupled to or associated with one or more input devices and one or more output devices and wherein the apparatuses, systems, and/or interfaces are configured to acquire user breathing data during an adverse breathing event and output audio recorded breathing patterns or rhythms, visual recorded breathing patterns or rhythms, audiovisual recorded breathing patterns or rhythms, and/or simulated/computer generated breathing patterns or rhythms to help the user recover from the adverse breathing event. In certain embodiments, the apparatuses, systems, and/or interfaces are configured to: (f) activate the apparatuses, systems, or interfaces (1) via the apparatuses, systems, and/or interfaces detecting an adverse breathing event or (2) via a user input; (g) acquire and/or receive initial breathing data from the user undergoing an adverse breathing event, (h) select a breathing output comprising audio recorded breathing pattern or rhythm, visual recorded breathing pattern or rhythm, audiovisual recorded breathing pattern or rhythm, and/or simulated/computer generated breathing pattern or rhythm (1) based on input from the user or (2) based on an automatic selection by the apparatuses, systems, and/or interfaces; (i) continue to acquire and/or receive breathing data from the user, (j) monitor the user breathing data, and (k) modify the breathing output based on the acquired breathing data. In other embodiments, the breathing output is selected to best assist the user in reestablishing a normal breathing pattern or rhythms based on the nature of the adverse breathing event the user is experiencing. In other embodiments, the patterns may also evidence differences between the user's current breathing pattern and the user's normal breathing pattern, wherein the differences may be evidenced by highlighting the differences either visually, acoustically, haptically, and/or any combination thereof. The highlighting may show the user's normal pattern with the user's current pattern in a superimposed format, with the differences highlighted so that as the pattern gets closer to the user's normal pattern, the highlighting gets less intense or fades as that user's breathing becomes more normal. The highlighting may be visual, auditory, audiovisual, and/or haptic and will change intensity or pulse rate, or twinkling as the user's breathing returns to normal.

Embodiments of this disclosure broadly relate to methods for implementing the breathing rhythm/pattern apparatuses, systems, and/or interfaces, wherein the apparatuses, systems, and/or interfaces include a processing unit having memory, communications hardware and software, one or more mass storage devices, and a power supply coupled to or associated with one or more input devices and one or more output devices and wherein the methods include acquiring user breathing data during an adverse breathing event and outputting audio recorded breathing patterns or rhythms, visual recorded breathing patterns or rhythms, audiovisual recorded breathing patterns or rhythms, and/or simulated/computer generated breathing patterns or rhythms to help the user recover from the adverse breathing event. In certain embodiments, the methods include: (f) activating the apparatuses, systems, and/or interfaces either (1) via the apparatuses, systems, and/or interfaces detecting an adverse breathing event or (2) via an input from the user; (g) acquiring and/or receiving initial breathing data from the user undergoing an adverse breathing event, (h) selecting a breathing output comprising audio recorded breathing pattern or rhythm, visual recorded breathing pattern or rhythm, audiovisual recorded breathing pattern or rhythm, and/or simulated/computer generated breathing pattern or rhythm (1) based on input from the user or (2) based on an automatic selection by the apparatuses, systems, and/or interfaces; (i) continue to acquiring and/or receiving breathing data from the user, (j) monitoring the user breathing data, and (k) modifying the breathing output based on the acquired breathing data. In other embodiments, the breathing output is selected to best assist the user in reestablishing a normal breathing pattern or rhythms based on the nature of the adverse breathing event the user is experiencing. In other embodiments, the method may also include the steps of evidencing differences between the user's current breathing pattern and the user's normal breathing pattern, wherein the differences may be evidenced by highlighting the differences either visually, acoustically, haptically, and/or any combination thereof. The highlighting may show the user's normal pattern with the user's current pattern in a superimposed format, with the differences highlighted so that as the pattern gets closer to the user's normal pattern, the highlighting gets less intense or fades as that user's breathing becomes more normal. The highlighting may be visual, auditory, audiovisual, and/or haptic and will change intensity or pulse rate, or twinkling as the user's breathing returns to normal.

The apparatuses or systems and/or the interfaces and/or methods implementing them may utilize any audio, visual, audiovisual, haptic, or other recording, recording sequences, or images or image sequence to be output in accord with acquired user breathing data to assist the user in reestablishing a normal breathing rhythm during an adverse breathing event or to assist the user in developing improved normal breathing rhythms.

The audio recordings or sequences thereof may include, without limitation, instrument recordings, songs, speeches, natural sounds (e.g., wind sounds, wave sounds, flowing water sounds, water fall sounds, rain sounds, storm sounds, any other natural sound, or any combination thereof), simulated sounds, augments natural sounds, human sounds, animal sounds, etc., or any combination thereof.

The visual recordings or sequences thereof may include, without limitation, nature images or image sequences (e.g., sky images, star images, planet images, moon images, galaxy images, galaxy cluster images, mountain images, hill images, plateau images, island images, continent images, sea images, lake images, river images, stream images, brook images, animal images, human images, any other natural image or sequence of images, or any combination thereof), simulated images, augments natural images, human images, animal images, etc., or any combination thereof.

The audiovisual recordings or sequences thereof may include, without limitation, nature images or image sequences (e.g., sky audiovisual recordings or sequences thereof, star audiovisual recordings or sequences thereof, planet recordings or sequences thereof, moon images, galaxy recordings or sequences thereof, galaxy cluster recordings or sequences thereof, mountain recordings or sequences thereof, hill recordings or sequences thereof, plateau recordings or sequences thereof, island recordings or sequences thereof, continent recordings or sequences thereof, sea recordings or sequences thereof, lake recordings or sequences thereof, river recordings or sequences thereof, stream recordings or sequences thereof, brook recordings or sequences thereof, animal recordings or sequences thereof, human recordings or sequences thereof, any other natural recordings or sequences thereof, or any combination thereof), simulated recordings or sequences thereof, augments natural recordings or sequences thereof, human recordings or sequences thereof, animal recordings or sequences thereof, etc., or any combination thereof.

The haptic recordings or sequences thereof may include, without limitation, nature haptic recordings or sequences thereof (e.g., heart beat haptic recordings or sequences thereof, breathing haptic recordings or sequences thereof, animal and/or human footfall haptic recordings or sequences thereof, rain haptic recordings or sequences thereof, wave haptic recordings or sequences thereof, falling water haptic recordings or sequences thereof, falling object haptic recordings or sequences thereof, any other haptic recordings or sequences thereof, or any combination thereof), simulated haptic recordings or sequences thereof, augments natural haptic recordings or sequences thereof, etc., or any combination thereof.

### Suitable Components for Use in the Disclosure

### Motion Sensors

Suitable motion sensors include, without limitation, optical sensors, acoustic sensors, thermal sensors, optoacoustic sensors, wave form sensors, pixel differentiation sensor, or any other sensor or combination of sensors that are capable of sensing movement or changes in movement, or mixtures and combinations thereof. Suitable motion sensing apparatus include, without limitation, motion sensors of any form such as digital cameras, optical scanners, optical roller ball devices, touch pads, inductive pads, capacitive pads, holographic devices, laser tracking devices, thermal devices, electromagnetic field (EMF) sensors, wave form sensors, any other device capable of sensing motion, changes in EMF, changes in a wave form, or the like or arrays of such devices or mixtures or combinations thereof. The sensors may be digital, analog, or a combination of digital and analog. The motion sensors may be touch pads, touchless pads, touch sensors, touchless sensors, inductive sensors, capacitive sensors, optical sensors, acoustic sensors, thermal sensors, optoacoustic sensors, electromagnetic field (EMF) sensors, strain gauges, accelerometers, pulse or waveform sensor, any other sensor that senses movement or changes in movement, or mixtures and combinations thereof. The sensors may be digital, analog, or a combination of digital and analog or any other type. For camera systems, the systems may sense motion within a zone, area, or volume in front of the lens or a plurality of lens. Optical sensors include any sensor using electromagnetic waves to detect movement or motion within in active zone. The optical sensors may operate in any region of the electromagnetic spectrum including, without limitation, radio frequency (RF), microwave, near infrared (IR), IR, far IR, visible, ultra violet (UV), or mixtures and combinations thereof. Exemplary optical sensors include, without limitation, camera systems, the systems may sense motion within a zone, area or volume in front of the lens. Acoustic sensor may operate over the entire sonic range which includes the human audio range, animal audio ranges, other ranges capable of being sensed by devices, or mixtures and combinations thereof. EMF sensors may be used and operate in any frequency range of the electromagnetic spectrum or any waveform or field sensing device that are capable of discerning motion with a given electromagnetic field (EMF), any other field, or combination thereof. Moreover, LCD screen(s), other screens and/or displays may be incorporated to identify which devices are chosen or the temperature setting, etc. Moreover, the interface may project a virtual control surface and sense motion within the projected image and invoke actions based on the sensed motion. The motion sensor associated with the interfaces of this disclosure may also be acoustic motion sensor using any acceptable region of the sound spectrum. A volume of a liquid or gas, where a user's body part or object under the control of a user may be immersed, may be used, where sensors associated with the liquid or gas can discern motion. Any sensor being able to discern differences in transverse, longitudinal, pulse, compression or any other waveform may be used to discern motion and any sensor measuring gravitational, magnetic, electro-magnetic, or electrical changes relating to motion or contact while moving (resistive and capacitive screens) could be used. Of course, the interfaces can include mixtures or combinations of any known or yet to be invented motion sensors. The motion sensors may be used in conjunction with displays, keyboards, touch pads, touchless pads, sensors of any type, or other devices associated with a computer, a notebook computer or a drawing tablet or any mobile or stationary device, and/or device, head worn device, or stationary device.

Suitable motion sensing apparatus include, without limitation, motion sensors of any form such as digital cameras, optical scanners, optical roller ball devices, touch pads, inductive pads, capacitive pads, holographic devices, laser tracking devices, thermal devices, electromagnetic field (EMF) sensors, wave form sensors, magnetic field (MEM) sensors, micro-electro-mechanical sensors, any other device capable of sensing motion, changes in EMF sensor reading, changes in wave form, or the like or arrays of such devices or mixtures or combinations thereof. Other motion sensors that sense changes in pressure, in stress and strain (strain gauges), changes in surface coverage measured by sensors that measure surface area or changes in surface are coverage, change in acceleration measured by accelerometers, or any other sensor that measures changes in force, pressure, velocity, volume, gravity, acceleration, any other force sensor or mixtures and combinations thereof.

### Controllable Objects

Suitable physical mechanical, electro-mechanical, magnetic, electro-magnetic, electrical, or electronic devices, hardware devices, appliances, biometric devices, automotive devices, VR objects, AR objects, MR objects, and/or any other real world device and/or virtual object that may be controlled by a processing unit include, without limitation, any electrical and/or hardware device or appliance or VR object that may or may not have attributes, all of which may be controlled by a switch, a joy stick, a stick controller, other similar type controller, and/or software programs or objects. Exemplary examples of such attributes include, without limitation, ON, OFF, intensity and/or amplitude, impedance, capacitance, inductance, software attributes, lists, submenus, layers, sublayers, other leveling formats associated with software programs, objects, haptic sensors and input devices, any other controllable electrical and/or electro-mechanical function and/or attribute of the device and/or mixtures or combinations thereof. Exemplary examples of devices include, without limitation, environmental controls, building systems and controls, lighting devices such as indoor and/or outdoor lights or light fixtures, cameras, ovens (conventional, convection, microwave, and/or etc.), dishwashers, stoves, sound systems, mobile devices, display systems (televisions (TVs), videocassette recorders (VCRs), digital video disc devices (DVDs), cable boxes, satellite boxes, and/or etc.), alarm systems, control systems, air conditioning systems (air conditions and heaters), energy management systems, medical devices, vehicles, robots, robotic control systems, unmanned aerial vehicle control (UAV) devices, equipment and machinery control systems, hot and cold water supply devices, air conditioning system, heating systems, fuel delivery systems, energy management systems, product delivery systems, ventilation systems, air handling systems, computers and computer systems, chemical plant control systems, manufacturing plant control systems, computer operating systems and other software systems, programs, routines, objects, and/or elements, remote control systems, or the like virtual and augmented reality systems, holograms, and/or mixtures or combinations thereof.

### Software Systems

Suitable software systems, software products, and/or software objects that are amenable to control by the interface of this disclosure include, without limitation, any analog or digital processing unit or units having single or a plurality of software products installed thereon and where each software product has one or more adjustable attributes associated therewith, or singular software programs or systems with one or more adjustable attributes, menus, lists, or other functions, attributes, and/or characteristics, and/or display outputs. Exemplary examples of such software products include, without limitation, operating systems, graphics systems, business software systems, word processor systems, business systems, online merchandising, online merchandising systems, purchasing and business transaction systems, databases, software programs and applications, internet browsers, accounting systems, military systems, control systems, VR, AR, MR systems or the like, or mixtures or combinations thereof. Software objects generally refer to all components within a software system or product that are controllable by at least one processing unit.

### Processing Units

Suitable processing units for use in the present disclosure include, without limitation, digital processing units (DPUs), analog processing units (APUs), Field Programmable Gate Arrays (FPGAs), any other technology that may receive motion sensor output and generate command and/or control functions for objects under the control of the processing unit, and/or mixtures and combinations thereof.

Suitable digital processing units (DPUs) include, without limitation, any digital processing unit capable of accepting input from a plurality of devices and converting at least some of the input into output designed to select and/or control attributes of one or more of the devices. Exemplary examples of such DPUs include, without limitation, microprocessor, microcontrollers, or the like manufactured by Intel, Motorola, Ericsson, HP, Samsung, Hitachi, NRC, Applied Materials, AMD, Cyrix, Sun Microsystem, Philips, National Semiconductor, Qualcomm, or any other manufacture of microprocessors or microcontrollers, and/or mixtures or combinations thereof.

Suitable analog processing units (APUs) include, without limitation, any analog processing unit capable of accepting input from a plurality of devices and converting at least some of the input into output designed to control attributes of one or more of the devices. Such analog devices are available from manufacturers such as Analog Devices Inc.

### User Feedback Units

Suitable user feedback units include, without limitation, cathode ray tubes, liquid crystal displays, light emitting diode displays, organic light emitting diode displays, plasma displays, touch screens, touch sensitive input/output devices, audio input/output devices, audio-visual input/output devices, holographic displays and environments, keyboard input devices, mouse input devices, optical input devices, and any other input and/or output device that permits a user to receive user intended inputs and generated output signals, and/or create input signals.

### Output and Input Devices

Suitable input and output devices for use herein include, without limitation, audio i/o devices such as speaker, visual i/o device such as displays, audiovisual i/o devices such as computers, laptops, tablets, phones, etc., haptic devices, EKG i/o devices, EEG i/o devices, heart rate monitoring i/o devices, breathing monitoring i/o devices, optical i/o devices, IR i/o devices, air flow i/o devices, thermal i/o devices, any other i/o device for monitoring human breathing and related phenomena, or any combination thereof.

### Predictive Breathing Simulation Methodology

The inventors have found that predictive virtual training systems, apparatuses, interfaces, and methods for implementing them may be constructed including one or more processing units, one or more motion sensing devices or motion sensors, optionally one or more non-motion sensors, one or more input devices, and one or more output devices such as one or more display devices, wherein the processing unit includes a virtual training program and is configured to (a) output the training program in response to user input data sensed by the sensors or received from the input devices, (b) collect user interaction data while performing the virtual training program, and (c) modify, alter, change, augment, update, enhance, reformat, restructure, and/or redesign the virtual training program to better tailor the virtual training program for each user, for each user, type and/or for all users.

### DETAILED DESCRIPTION OF THE DRAWINGS OF THE DISCLOSURE

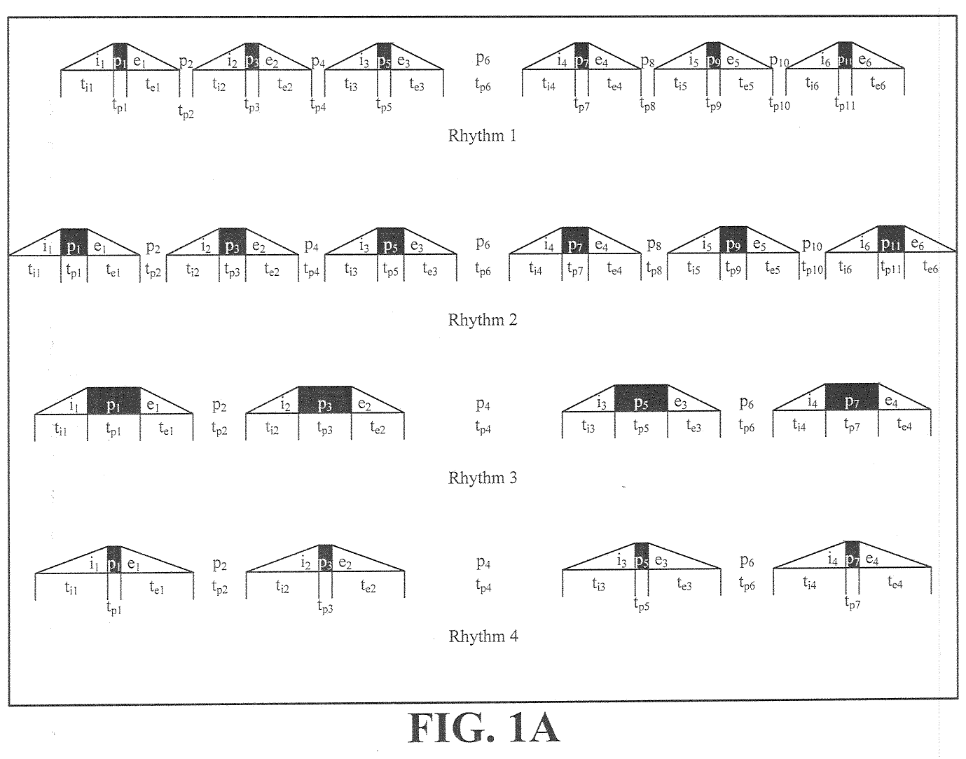

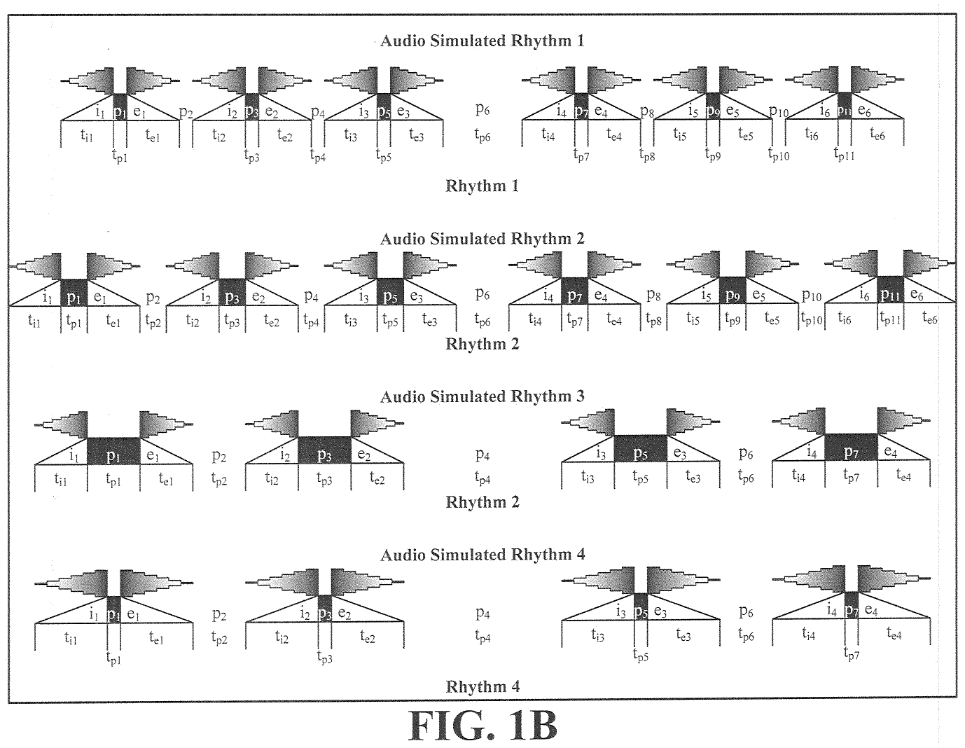

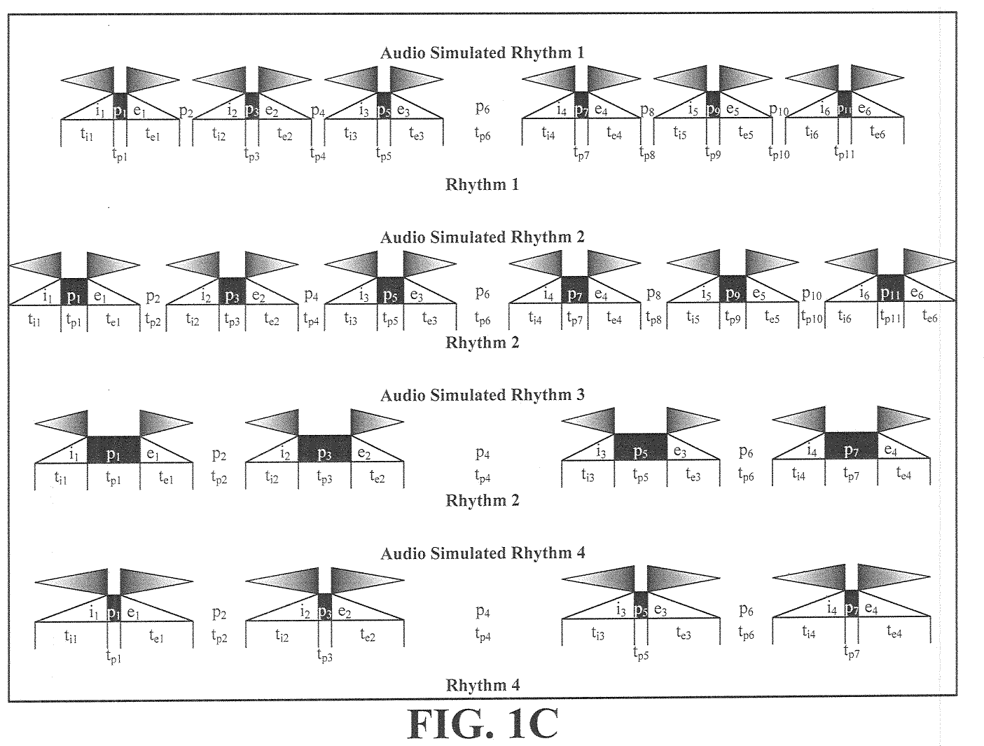

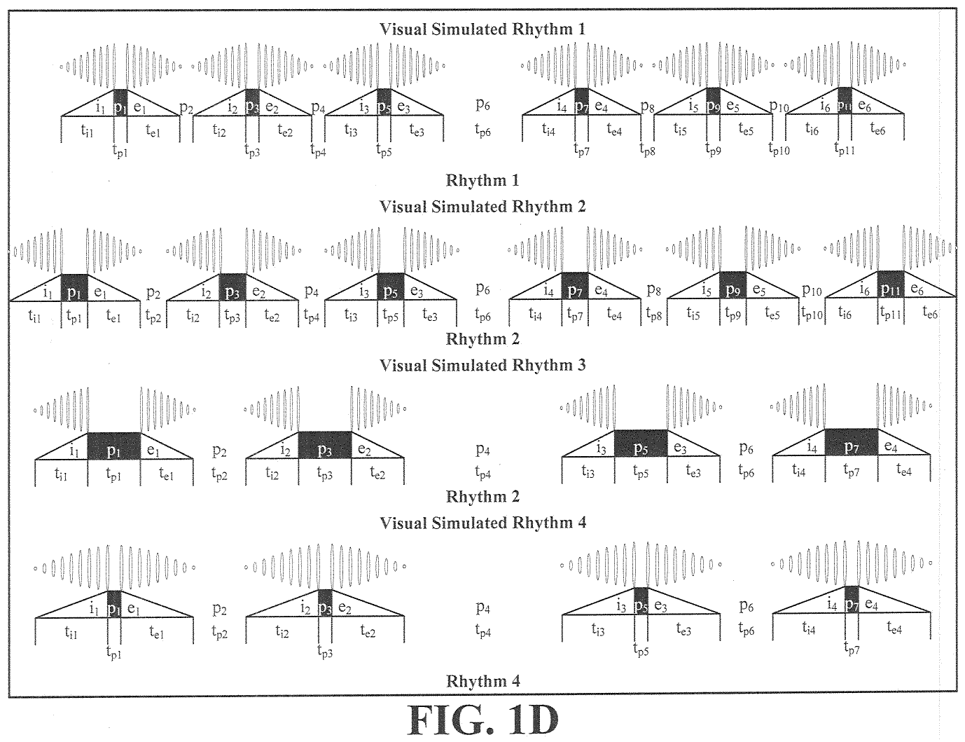

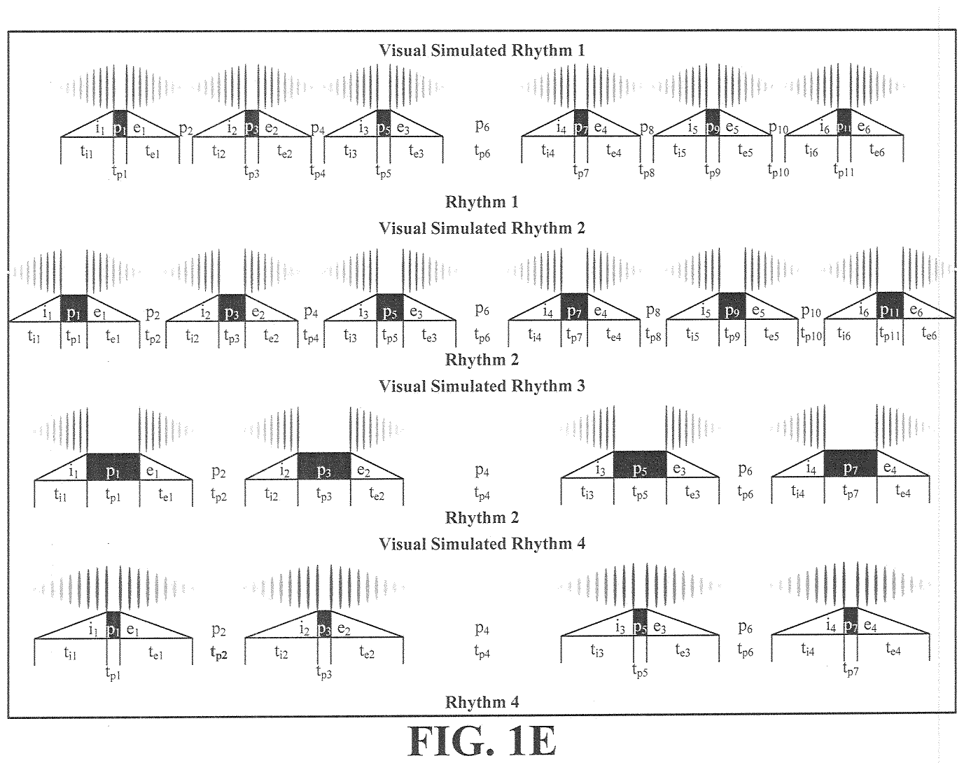

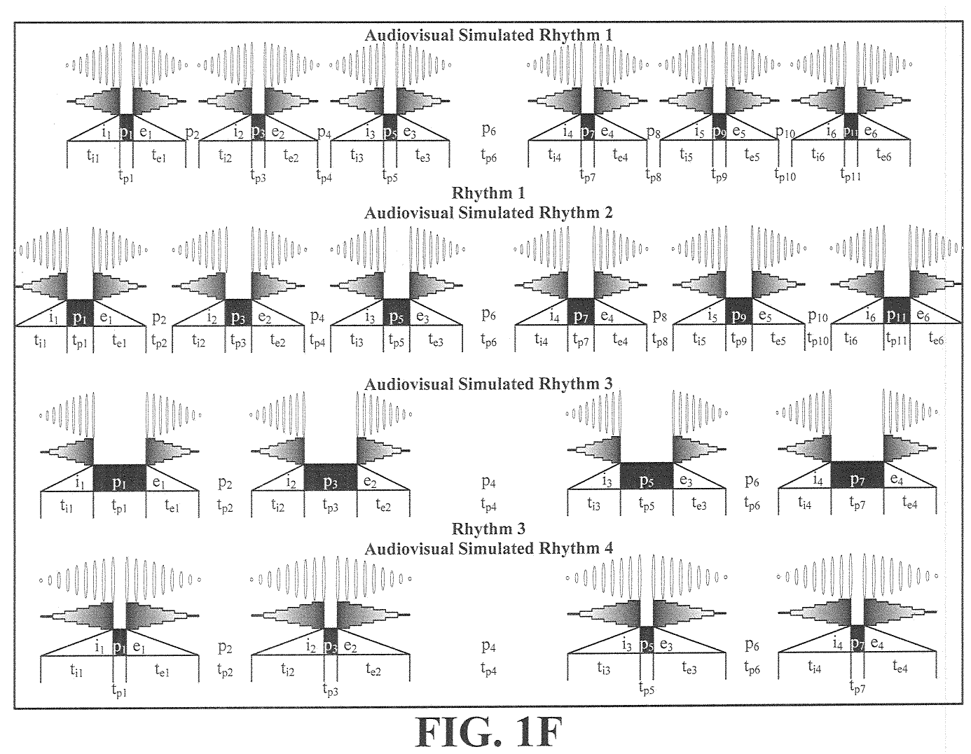

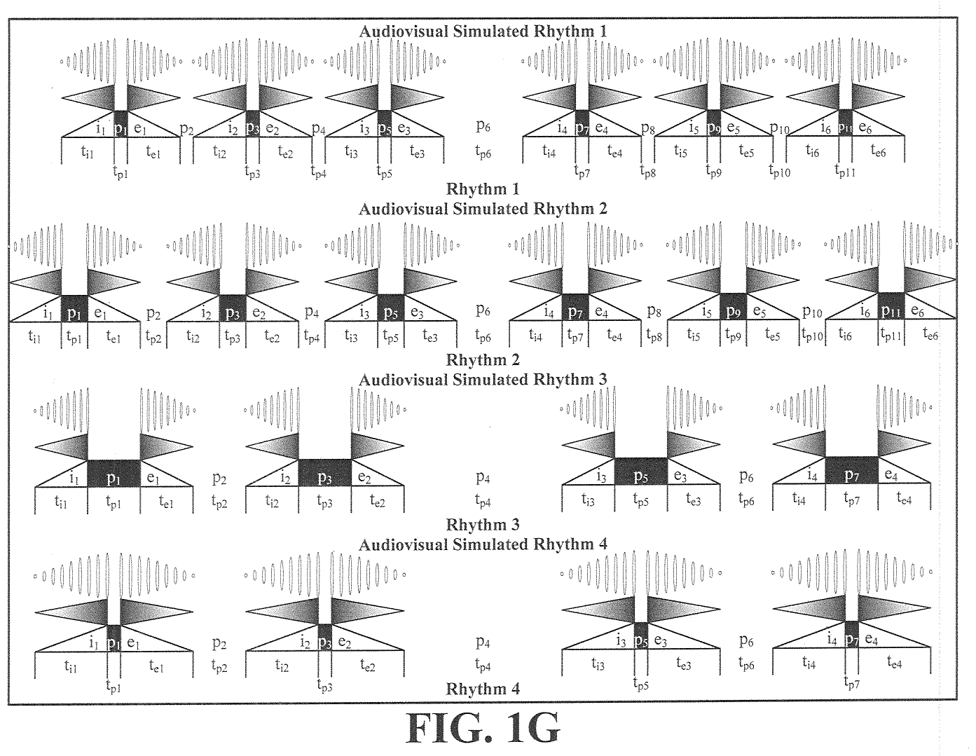

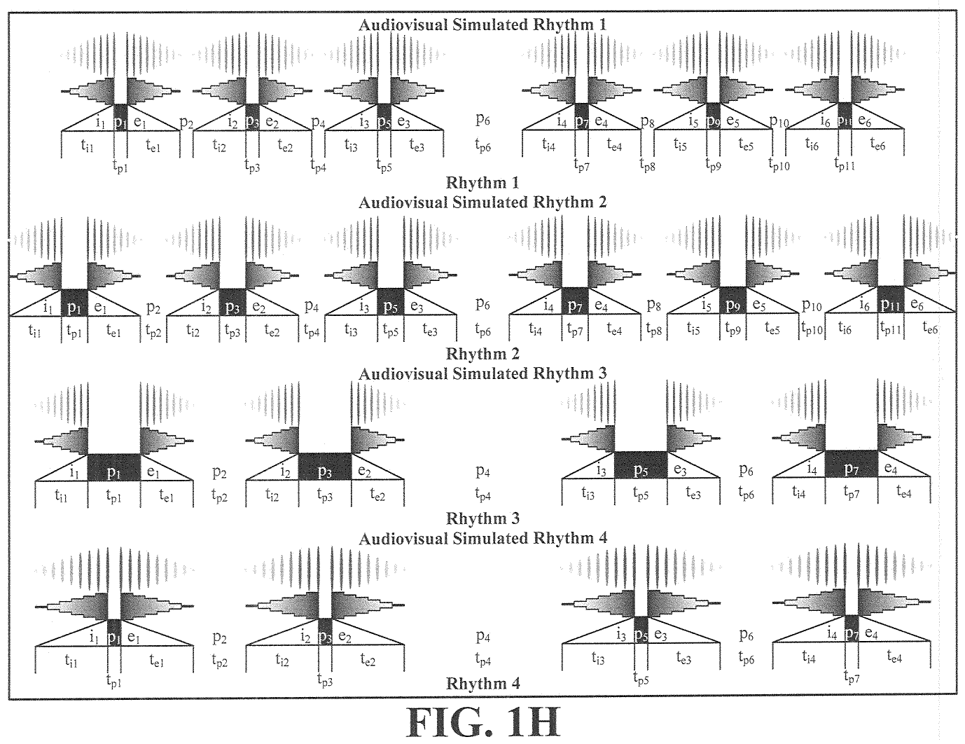

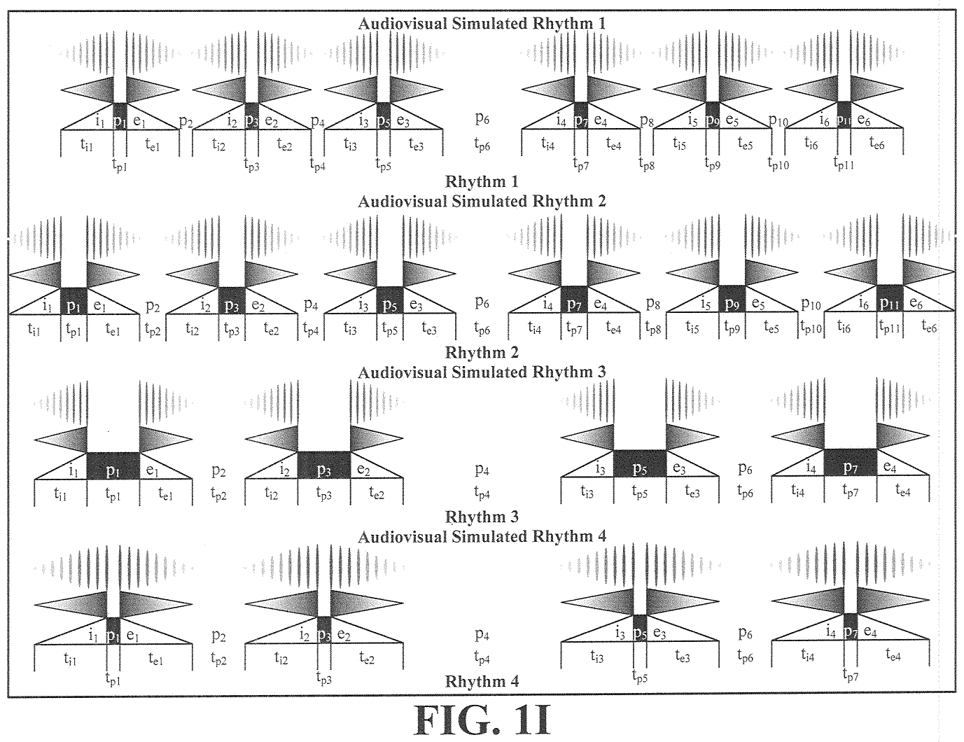

FIG. 1A depicts several breathing rhythms, Rhythms 1-4, which are different known standard breathing rhythms or patterns.

The Rhythm 1 comprises: (a) a first breadth including a first inhalation i₁ of duration tᵢ₁, a first pause p₁ of duration tₚ₁, a first exhalation e₁ of duration tₑ₁ followed by a second pause p₂ of duration tₚ₂; (b) a second breadth including a second inhalation i₂ of duration tᵢ₂, a third pause p₃ of duration tₚ₃; a second exhalation e₂ of duration tₑ₂ followed by a fourth pause p₄ of duration tₚ₄; (c) a third breadth including a third inhalation i₃ of duration tᵢ₃, a fifth pause p₅ of duration tₚ₅, a third exhalation e₃ of duration tₑ₃ followed by a sixth pause p₆ of duration tₚ₁; (d) a fourth breadth including a fourth inhalation i₄ of duration tᵢ₄, a seventh pause p₇ of duration tₚ₇, a fourth exhalation e₄ of duration tₑ₄ followed by a eighth pause p₈ of duration tₚ₈; (e) a fifth breadth including a fifth inhalation i₅ of duration tᵢ₅, a ninth pause p₉ of duration tₚ₉; a fifth exhalation e₅ of duration tₑ₅ followed by a tenth pause p₁₀ of duration tₚ₁₀; and (f) a sixth breadth including a sixth inhalation i₆ of duration tᵢ₂, an eleventh pause p₁₁ of duration tₚ₁₁, a sixth exhalation e₆ of duration tₑ₆.

The Rhythm 2 comprises: (a) a first breadth including a first inhalation i₁ of duration tᵢ₁, a first pause p₁ of duration tₚ₁, a first exhalation e₁ of duration tₑ₁ followed by a second pause p₂ of duration tₚ₂; (b) a second breadth including a second inhalation i₂ of duration tᵢ₂, a third pause p₃ of duration tₚ₃; a second exhalation e₂ of duration tₑ₂ followed by a fourth pause p₄ of duration tₚ₄; (c) a third breadth including a third inhalation i₃ of duration tᵢ₃, a fifth pause p₅ of duration tₚ₅; a third exhalation e₃ of duration tₑ₃ followed by a sixth pause p₆ of duration tₚ₆; (d) a fourth breadth including a fourth inhalation i₄ of duration tᵢ₄, a seventh pause p₇ of duration tₚ₇, a fourth exhalation e₄ of duration tₑ₄ followed by a eighth pause p₈ of duration tₚ₈; (e) a fifth breadth including a fifth inhalation i₅ of duration t₅, a ninth pause p₉ of duration tₚ₉; a fifth exhalation e₅ of duration tₑ₅ followed by a tenth pause p₁₀ of duration tₚ₁₀; and (f) a sixth breadth including a sixth inhalation i₆ of duration tᵢ₂, an eleventh pause p₁₁ of duration tₚ₁₁, a sixth exhalation e₆ of duration tₑ₆, wherein the pauses p₁ through p₅ and p₇ through p₁₁ are longer and p₆ is shorter.

The Rhythm 3 comprises: (a) a first breadth including a first inhalation i₁ of duration tᵢ₁, a first pause p₁ of duration tₚ₁, a first exhalation e₁ of duration tₑ₁ followed by a second pause p₂ of duration tₚ₂; (b) a second breadth including a second inhalation i₂ of duration t₁₂, a third pause p₃ of duration tₚ₃; a second exhalation e₂ of duration tₑ₂ followed by a fourth pause p₄ of duration tₚ₄; (c) a third breadth including a third inhalation i₃ of duration tᵢ₃, a fifth pause p₅ of duration tₚ₅; a third exhalation e₃ of duration tₑ₃ followed by a sixth pause p₆ of duration tₚ₆; and (d) a fourth breadth including a fourth inhalation i₄ of duration tᵢ₄, a seventh pause p₇ of duration tₚ₇, a fourth exhalation e₄ of duration tₑ₄ followed by a eighth pause p₈ of duration tₚ₈; wherein p₂, p₃, p₄, p₅, and p₆ are even longer.

The Rhythm 4 comprises: (a) a first breadth including a first inhalation i₁ of duration tᵢ₁, a first pause p₁ of duration tₚ₁, a first exhalation e₁ of duration tₑ₁ followed by a second pause p₂ of duration tₚ₂; (b) a second breadth including a second inhalation i₂ of duration tᵢ₂, a third pause p₃ of duration tₚ₃; a second exhalation e₂ of duration tₑ₂ followed by a fourth pause p₄ of duration tₚ₄; (c) a third breadth including a third inhalation i₃ of duration tᵢ₃, a fifth pause p₅ of duration tₚ₅, a third exhalation e₃ of duration tₑ₃ followed by a sixth pause p₆ of duration tₚ₆; and (d) a fourth breadth including a fourth inhalation i₄ of duration tᵢ₄, a seventh pause p₇ of duration tₚ₇, a fourth exhalation e₄ of duration tₑ₄ followed by a eighth pause p₈ of duration tₚ₈; wherein i₁ through i₄ and e₁ through e₄ are longer, while p₁, p₃, p₅, and p₇ are shorter.

It should be recognized that the durations on any of the rhythms may be same or different so that they correspond to known breathing rhythms and patterns. It should also be recognized that these durations may be used to simulate irregular breathing rhythms or patterns that accompany an adverse breathing event.

FIG. 1B depicts Rhythms 1-4 including inhalation audio recordings or simulations that rise in volume with inhalations in inhalation stepwise fashions and exhalation audio recordings or simulations that lower in volume with exhalations in exhalation stepwise fashions, wherein the inhalation audio recordings or simulations and the exhalation audio recordings or simulations may be the same or different and the inhalation stepwise fashions and the exhalation stepwise fashions are same or different manner.

FIG. 1C depicts Rhythms 1-4 including inhalation audio recordings or simulations that rise in volume with inhalations in inhalation continuous fashions and exhalation audio recordings or simulations that lower in volume with exhalations in exhalation continuous fashions, wherein the inhalation audio recordings or simulations and the exhalation audio recordings or simulations may be the same or different and the inhalation stepwise fashions and the exhalation stepwise fashions are same or different manner.

FIG. 1D depicts Rhythms 1-4 including inhalation visual recordings or simulations having inhalation objects that increase with inhalations in inhalation fashions and exhalation visual recordings or simulations having exhalation objects that decrease with exhalations in exhalation fashions, wherein the inhalation objects and the exhalation objects may be the same or different, the inhalation visual recordings or simulations and the exhalation visual recordings or simulations may be the same or different, and the inhalation fashions and the exhalation fashions may be the same or different. The inhalation objects and the exhalation objects are shown here as gray scale ellipses that increase in size with inhalations and decrease in size with exhalations.

FIG. 1E depicts Rhythms 1-4 including inhalation visual recordings or simulations having inhalation objects that increase with inhalations in inhalation fashions and exhalation visual recordings or simulations having exhalation objects that decrease with exhalations in exhalation fashions, wherein the inhalation objects and the exhalation objects may be the same or different, the inhalation visual recordings or simulations and the exhalation visual recordings or simulations may be the same or different, and the inhalation fashions and the exhalation fashions may be the same or different. The inhalation objects and the exhalation objects are shown here as colored (pink) scale ellipses that increase in size with inhalations and decrease in size with exhalations.

FIG. 1F depicts Rhythms 1-4 including inhalation audio recordings or simulations that rise in volume with inhalations in inhalation stepwise fashions and exhalation audio recordings or simulations that lower in volume with exhalations in exhalation stepwise fashions, wherein the inhalation audio recordings or simulations and the exhalation audio recordings or simulations may be the same or different and the inhalation stepwise fashions and the exhalation stepwise fashions are same or different manner. The Rhythms 1-4 also include inhalation visual recordings or simulations having inhalation objects that increase with inhalations in inhalation fashions and exhalation visual recordings or simulations having exhalation objects that decrease with exhalations in exhalation fashions, wherein the inhalation objects and the exhalation objects may be the same or different, the inhalation visual recordings or simulations and the exhalation visual recordings or simulations may be the same or different, and the inhalation fashions and the exhalation fashions may be the same or different. The inhalation objects and the exhalation objects are shown here as gray scale ellipses that increase in size with inhalations and decrease in size with exhalations.

FIG. 1G depicts Rhythms 1-4 including inhalation audio recordings or simulations that rise in volume with inhalations in inhalation continuous fashions and exhalation audio recordings or simulations that lower in volume with exhalations in exhalation continuous fashions, wherein the inhalation audio recordings or simulations and the exhalation audio recordings or simulations may be the same or different and the inhalation stepwise fashions and the exhalation stepwise fashions are same or different manner. The Rhythms 1-4 also include inhalation visual recordings or simulations having inhalation objects that increase with inhalations in inhalation fashions and exhalation visual recordings or simulations having exhalation objects that decrease with exhalations in exhalation fashions, wherein the inhalation objects and the exhalation objects may be the same or different, the inhalation visual recordings or simulations and the exhalation visual recordings or simulations may be the same or different, and the inhalation fashions and the exhalation fashions may be the same or different. The inhalation objects and the exhalation objects are shown here as colored (pink) scale ellipses that increase in size with inhalations and decrease in size with exhalations.

FIG. 1H depicts Rhythms 1-4 including inhalation audio recordings or simulations that rise in volume with inhalations in inhalation stepwise fashions and exhalation audio recordings or simulations that lower in volume with exhalations in exhalation stepwise fashions, wherein the inhalation audio recordings or simulations and the exhalation audio recordings or simulations may be the same or different and the inhalation stepwise fashions and the exhalation stepwise fashions are same or different manner. The Rhythms 1-4 also include inhalation visual recordings or simulations having inhalation objects that increase with inhalations in inhalation fashions and exhalation visual recordings or simulations having exhalation objects that decrease with exhalations in exhalation fashions, wherein the inhalation objects and the exhalation objects may be the same or different, the inhalation visual recordings or simulations and the exhalation visual recordings or simulations may be the same or different, and the inhalation fashions and the exhalation fashions may be the same or different. The inhalation objects and the exhalation objects are shown here as gray scale ellipses that increase in size with inhalations and decrease in size with exhalations.

FIG. 1I depicts Rhythms 1-4 including inhalation audio recordings or simulations that rise in volume with inhalations in inhalation continuous fashions and exhalation audio recordings or simulations that lower in volume with exhalations in exhalation continuous fashions, wherein the inhalation audio recordings or simulations and the exhalation audio recordings or simulations may be the same or different and the inhalation stepwise fashions and the exhalation stepwise fashions are same or different manner. The Rhythms 1-4 also include inhalation visual recordings or simulations having inhalation objects that increase with inhalations in inhalation fashions and exhalation visual recordings or simulations having exhalation objects that decrease with exhalations in exhalation fashions, wherein the inhalation objects and the exhalation objects may be the same or different, the inhalation visual recordings or simulations and the exhalation visual recordings or simulations may be the same or different, and the inhalation fashions and the exhalation fashions may be the same or different. The inhalation objects and the exhalation objects are shown here as colored (pink) scale ellipses that increase in size with inhalations and decrease in size with exhalations.

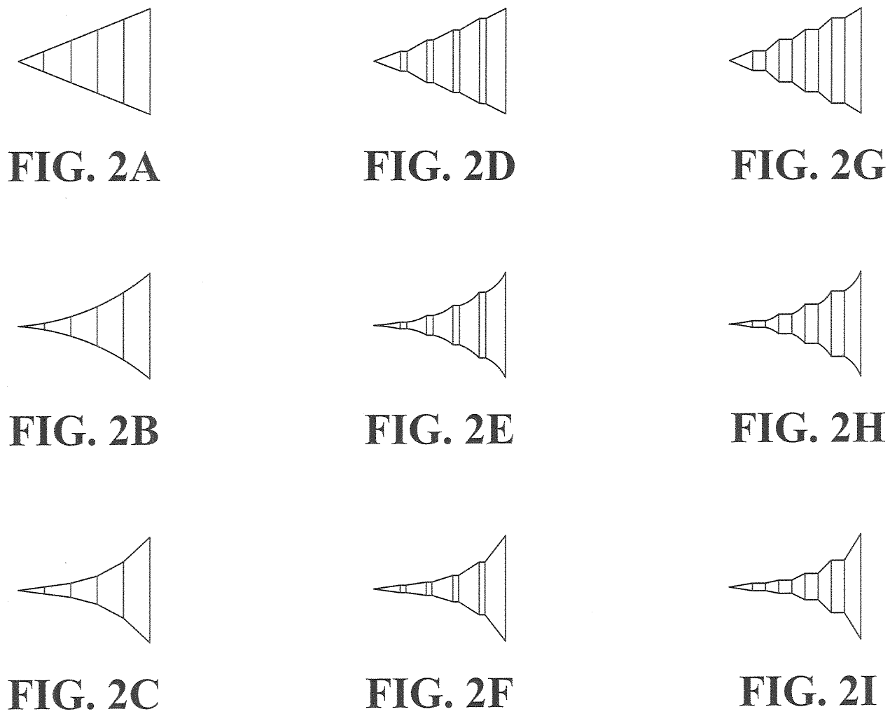

FIG. 2A-I depict different continuation audio recording or simulation formats some including volume increases and volume pauses or volume decreases and volume pauses.

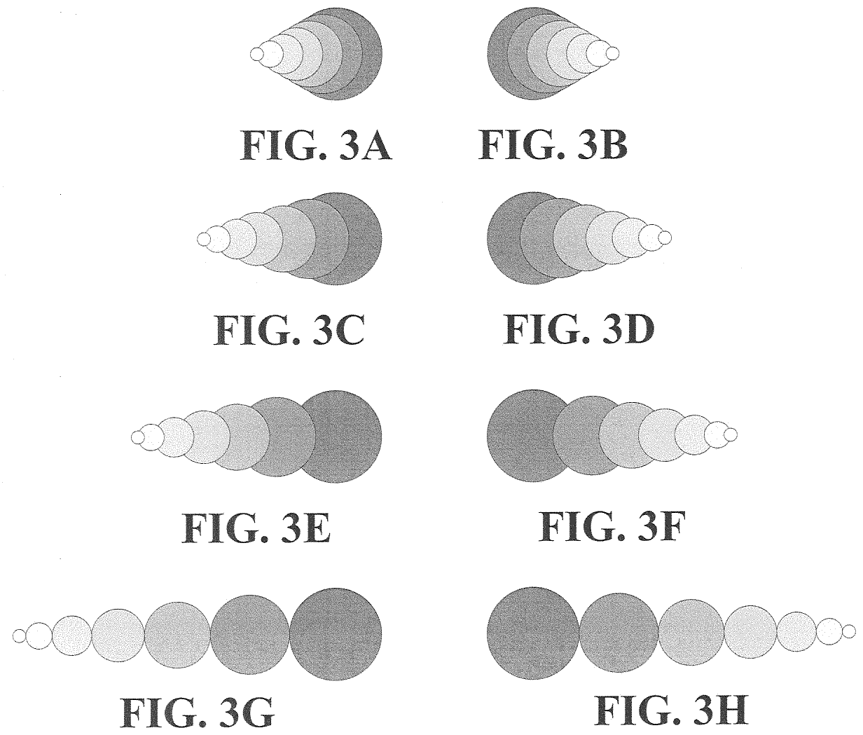

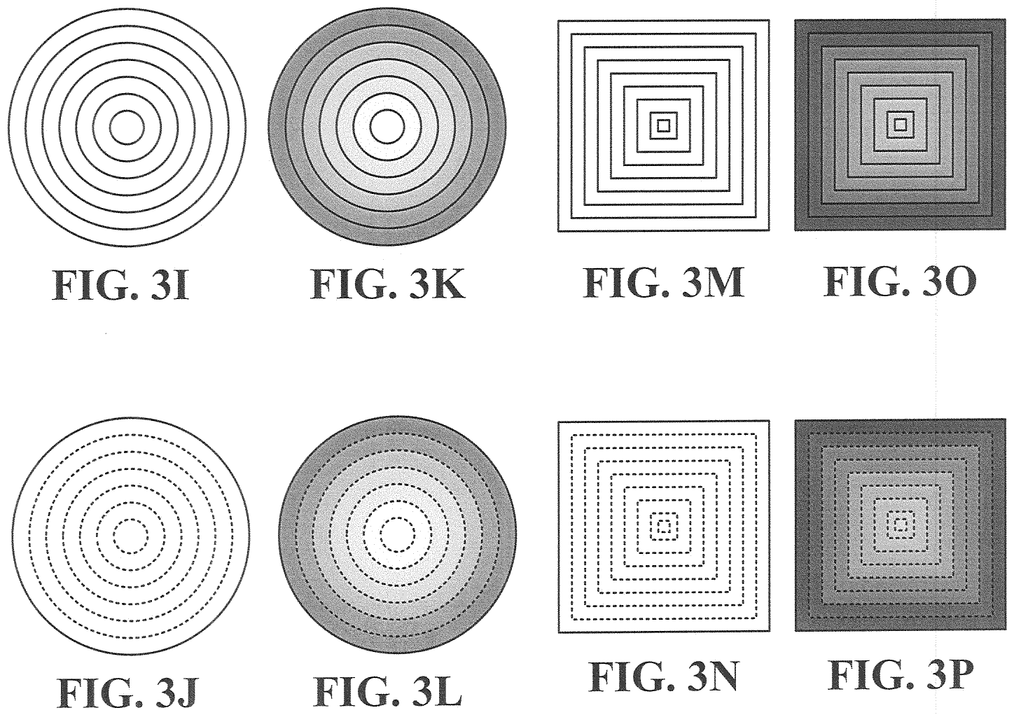

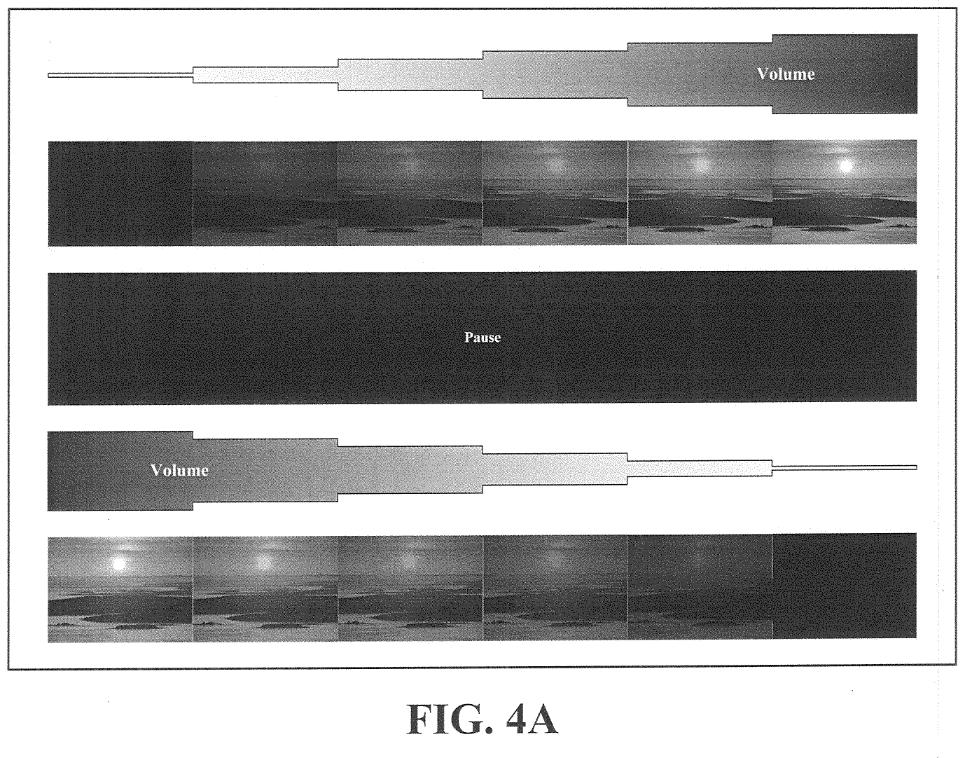

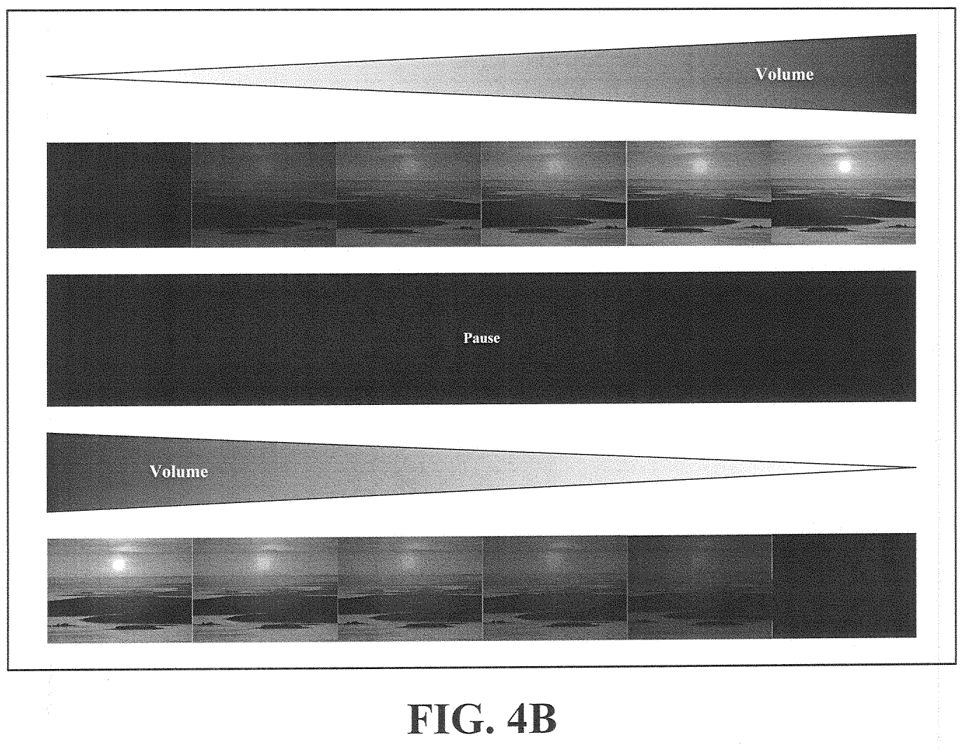

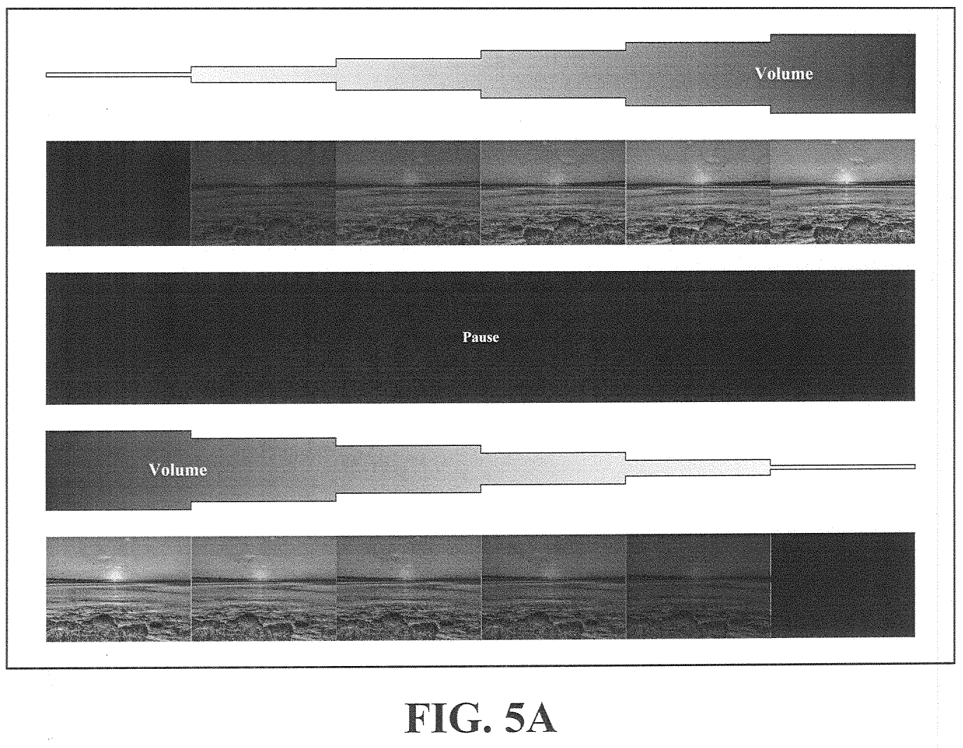

FIG. 3A-H depict different visual recordings and simulations involving grey scale circles in different arrangements.

FIG. 3I-J depict different visual recordings and simulations involving circles that increase during inhalations or decrease during exhalations.

FIG. 3K-L depict different visual recordings and simulations involving grey scale circles that increase during inhalations or decrease during exhalations.

FIG. 3M-N depict different visual recordings and simulations involving squares that increase during inhalations or decrease during exhalations.

FIG. 3O-P depict different visual recordings and simulations involving color scale squares that increase during inhalations or decrease during exhalations, while the image is in black and white, the square may be any color with shading from darker to lighter as the squares get smaller or from light to dark as the squares get smaller depending on whether the square represent breathing in or breathing out, where the shading changes as in accord with the user's current breathing pattern or the user's normal breathing pattern or evidences a difference between the user's current breathing pattern or the user's normal breathing pattern by highlighting either visually, acoustically, or a combination of both visually and acoustically.

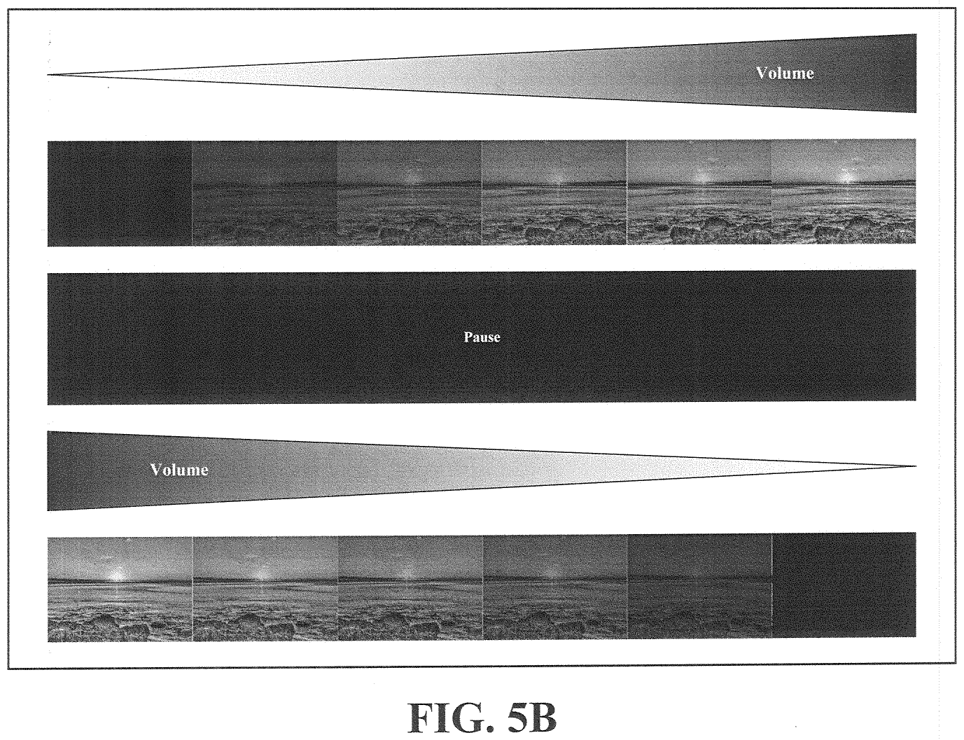

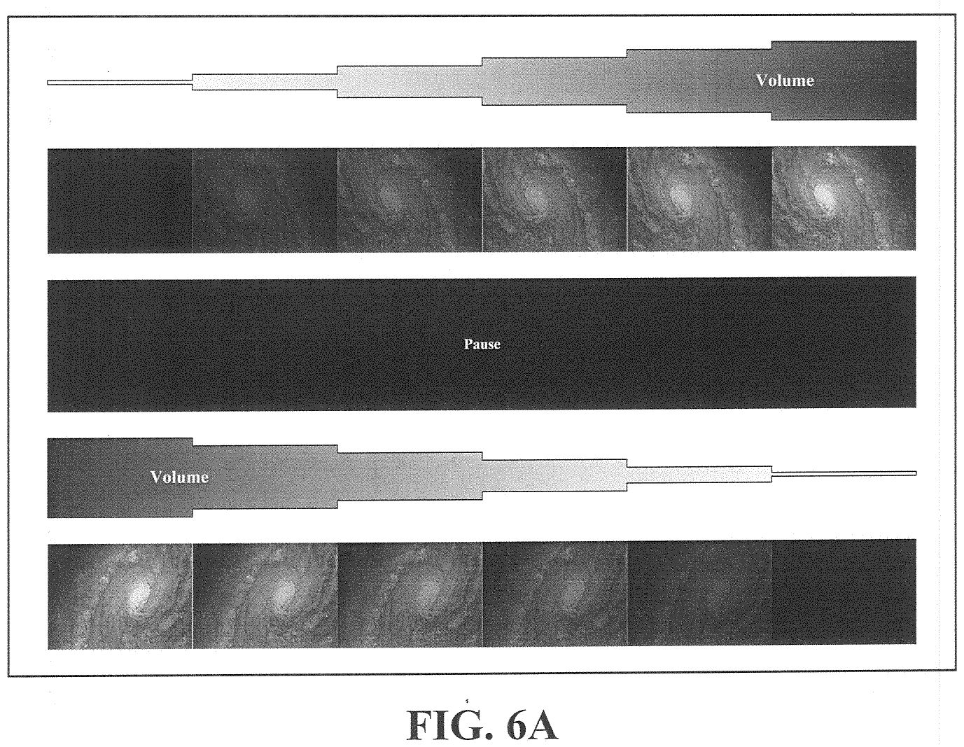

FIG. 4A depicts an inhalation and exhalation simulation including stepwise audio recordings or simulations and a stepwise sunset visualization that grows in intensity during inhalation and fades in intensity during exhalation.

FIG. 4B depicts an inhalation and exhalation simulation including continuous audio recordings or simulations and a stepwise the sunset visualization that grows in intensity during inhalation and fades in intensity during exhalation.

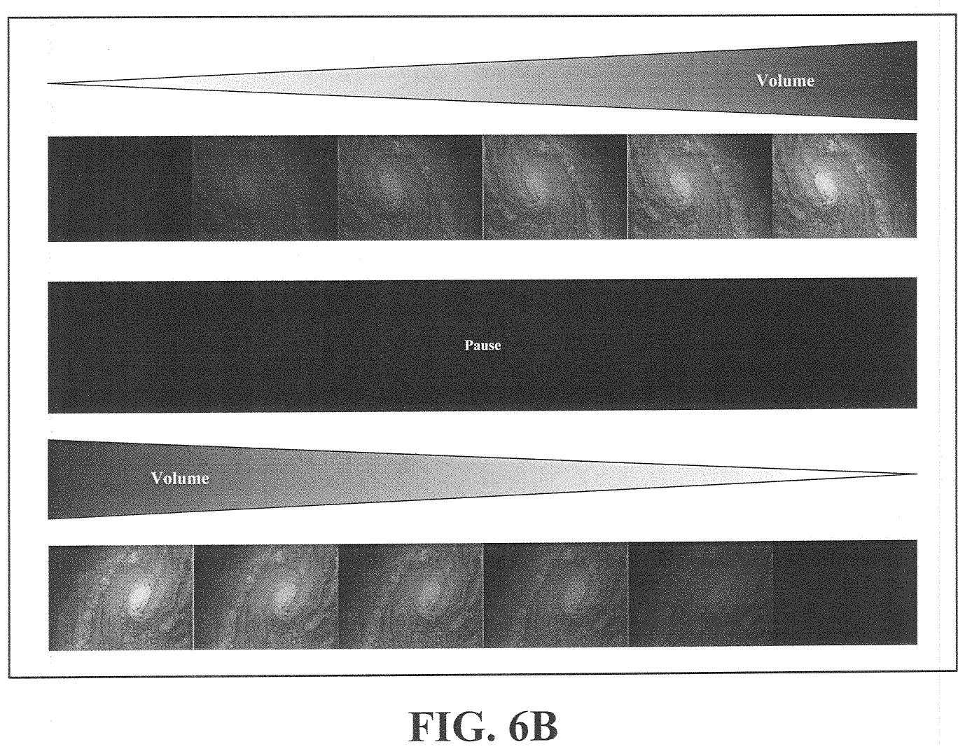

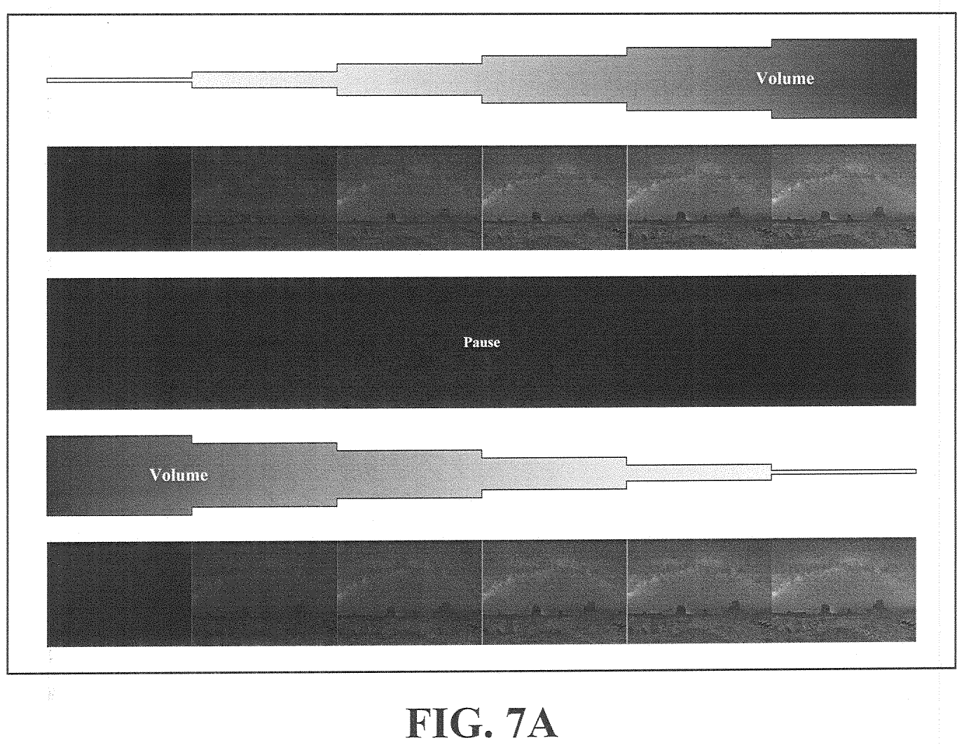

FIG. 5A depicts an inhalation and exhalation simulation including stepwise audio recordings or simulations and a stepwise sunrise visualization that grows in intensity during inhalation and fades in intensity during exhalation.

FIG. 5B depicts an inhalation and exhalation simulation including continuous audio recordings or simulations and a stepwise the sunrise visualization that grows in intensity during inhalation and fades in intensity during exhalation.

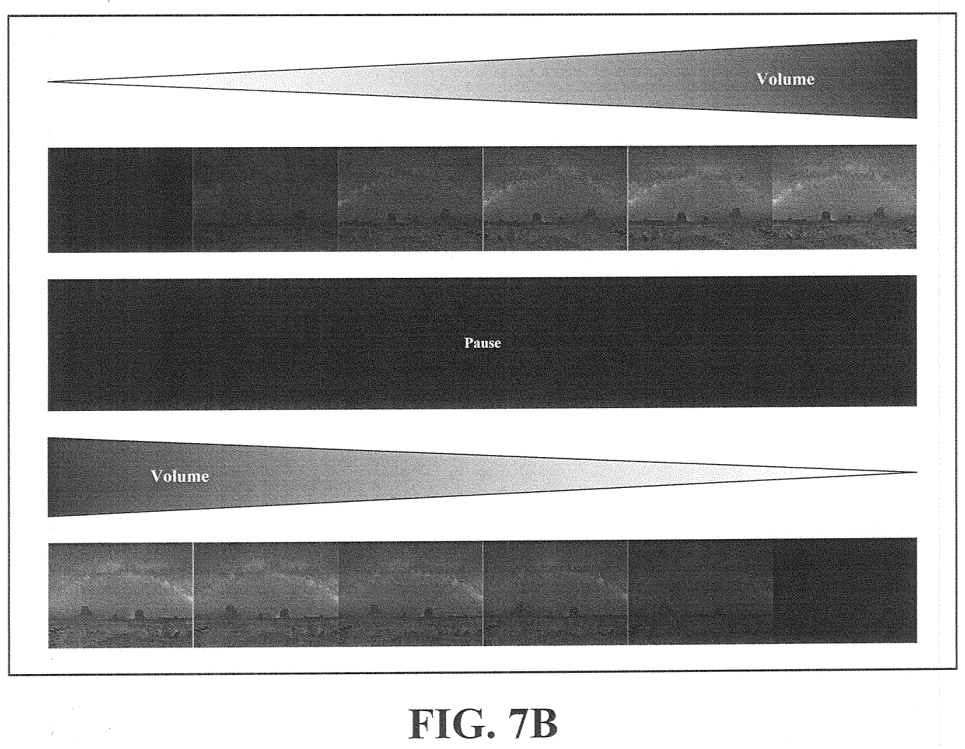

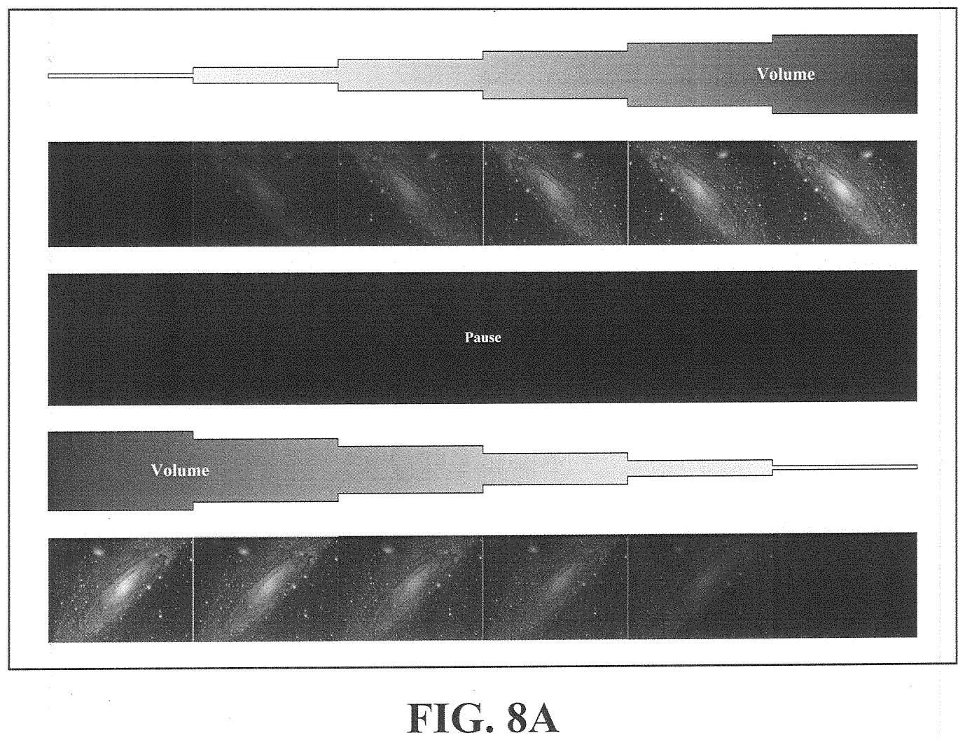

FIG. 6A depicts an inhalation and exhalation simulation including stepwise audio recordings or simulations and a stepwise spiral galaxy visualization that grows in intensity during inhalation and fades in intensity during exhalation.

FIG. 6B depicts an inhalation and exhalation simulation including continuous audio recordings or simulations and a stepwise the spiral galaxy visualization that grows in intensity during inhalation and fades in intensity during exhalation.

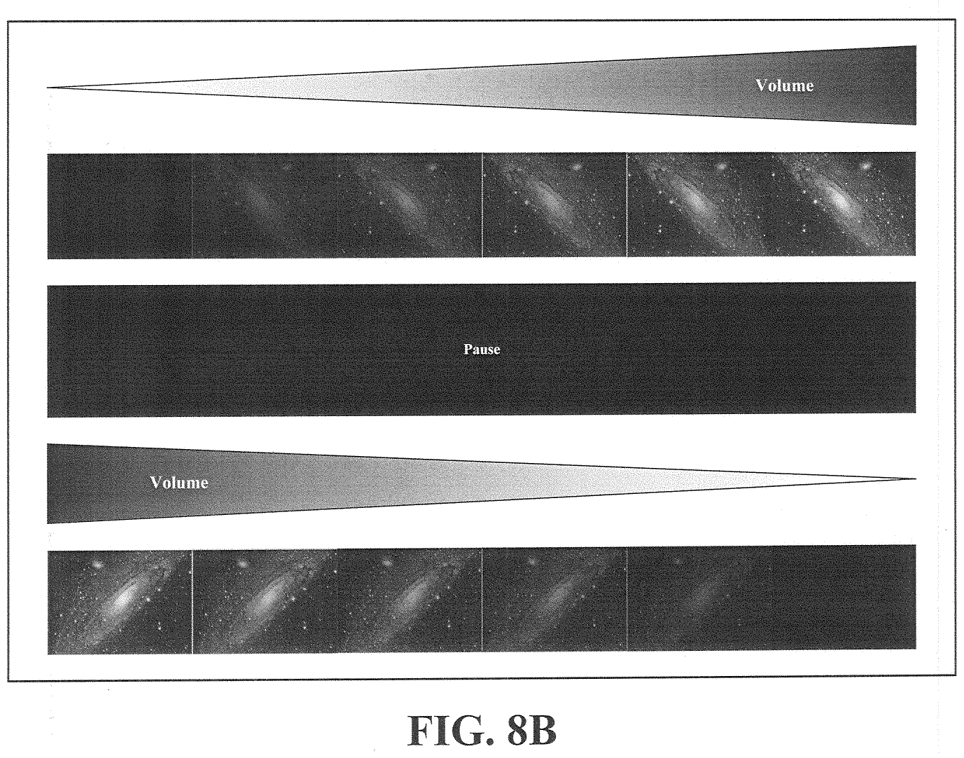

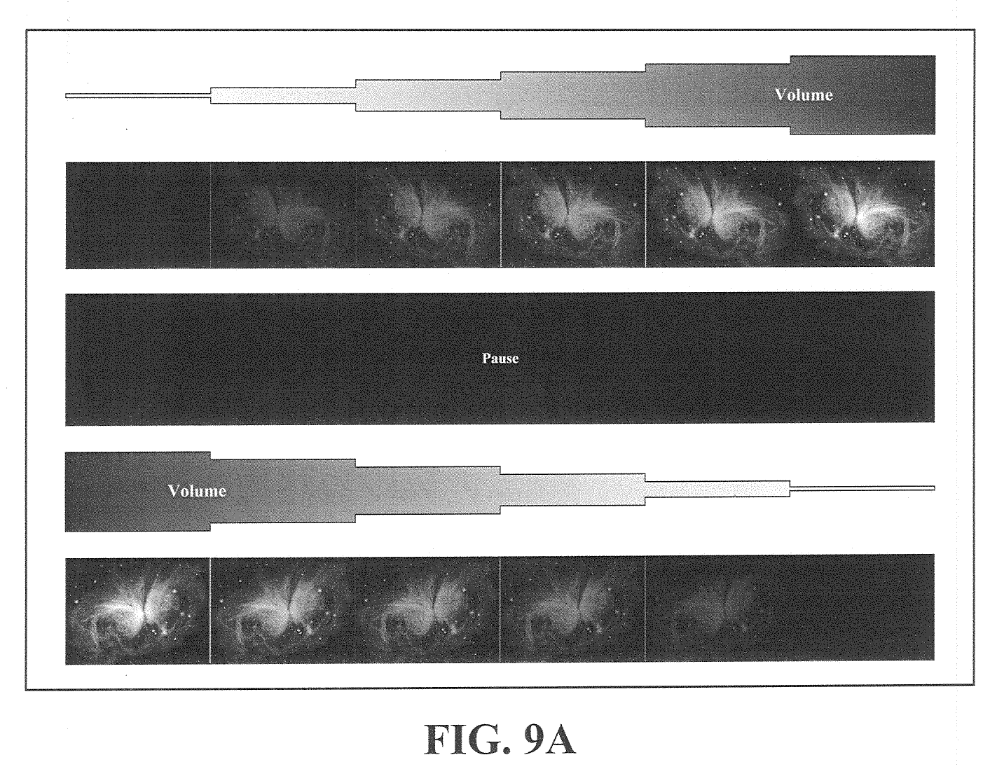

FIG. 7A depicts an inhalation and exhalation simulation including stepwise audio recordings or simulations and a stepwise milkway galaxy visualization that grows in intensity during inhalation and fades in intensity during exhalation.

FIG. 7B depicts an inhalation and exhalation simulation including continuous audio recordings or simulations and a stepwise the milkway galaxy visualization that grows in intensity during inhalation and fades in intensity during exhalation.

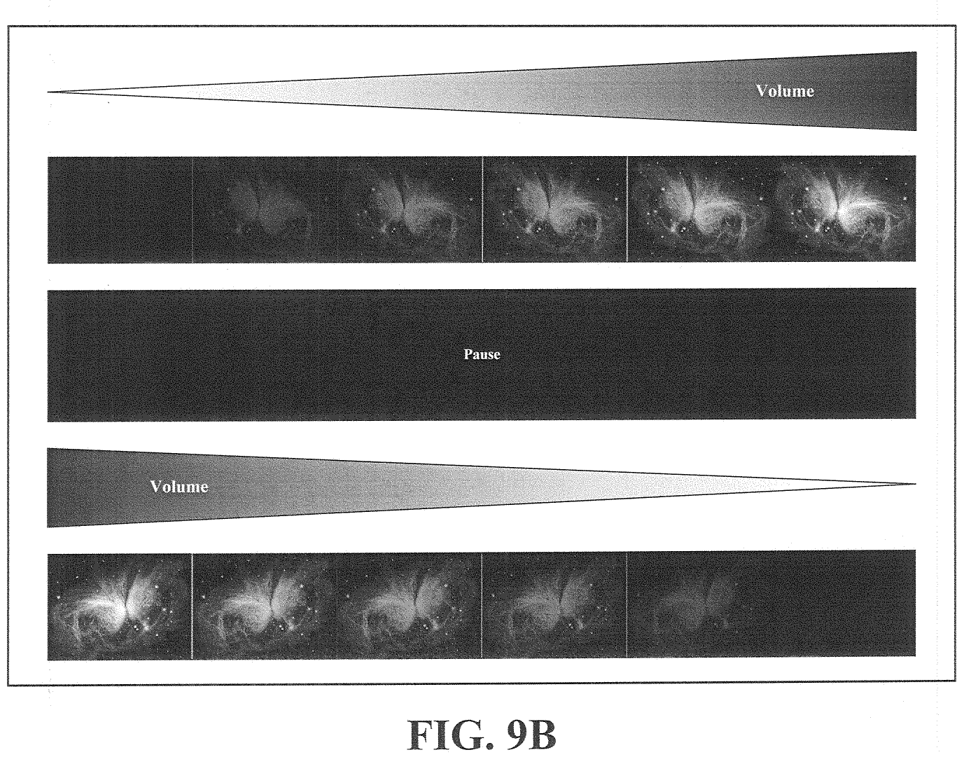

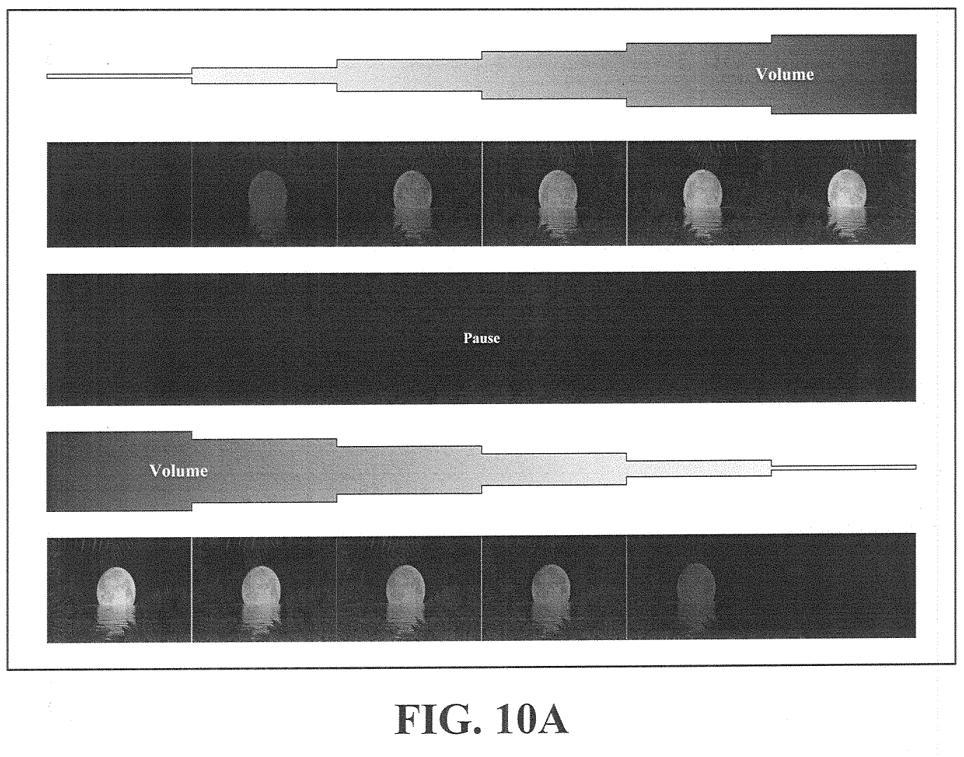

FIG. 8A depicts an inhalation and exhalation simulation including stepwise audio recordings or simulations and a stepwise Andromeda galaxy visualization that grows in intensity during inhalation and fades in intensity during exhalation.

FIG. 8B depicts an inhalation and exhalation simulation including continuous audio recordings or simulations and a stepwise the Andromeda galaxy visualization that grows in intensity during inhalation and fades in intensity during exhalation.

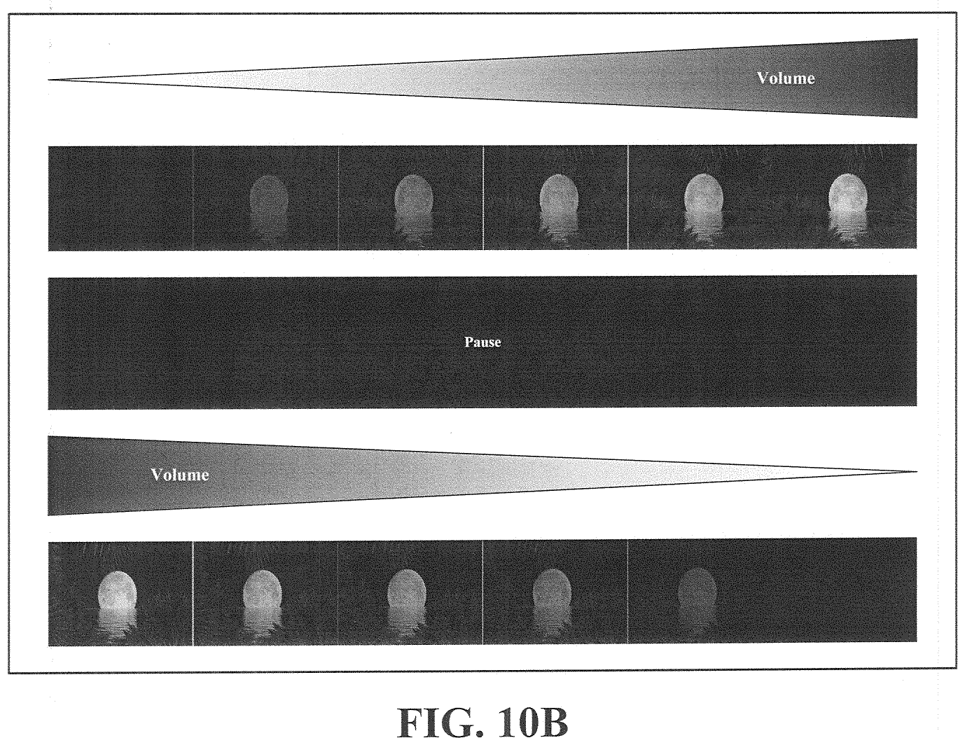

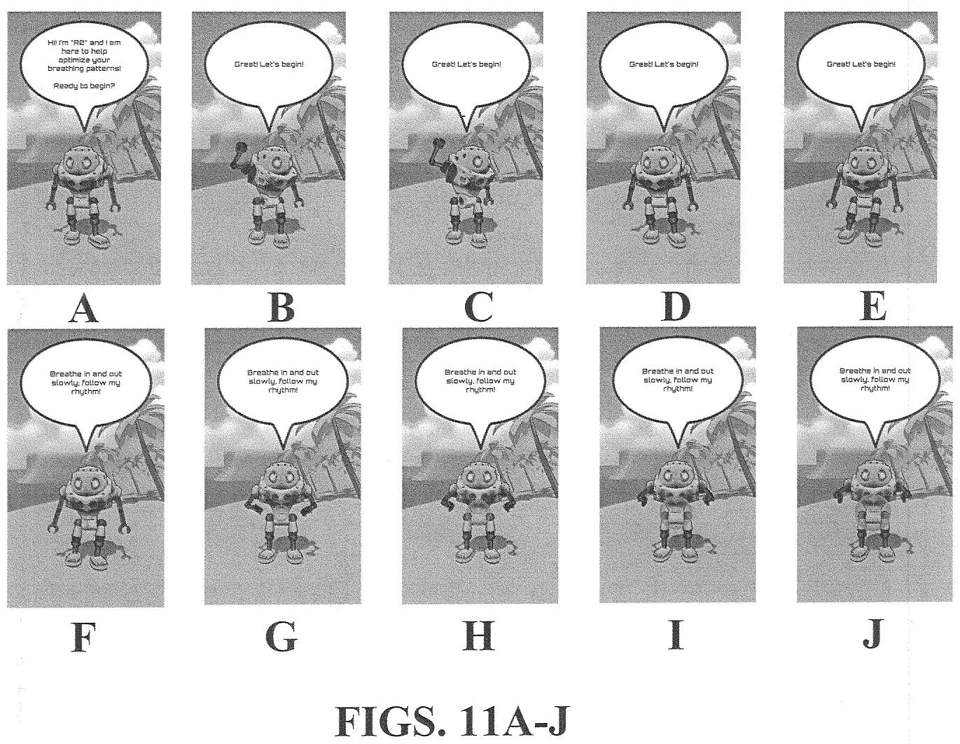

FIG. 9A depicts an inhalation and exhalation simulation including stepwise audio recordings or simulations and a stepwise Hubble star event visualization that grows in intensity during inhalation and fades in intensity during exhalation.

FIG. 9B depicts an inhalation and exhalation simulation including continuous audio recordings or simulations and a stepwise the Hubble star event visualization that grows in intensity during inhalation and fades in intensity during exhalation.

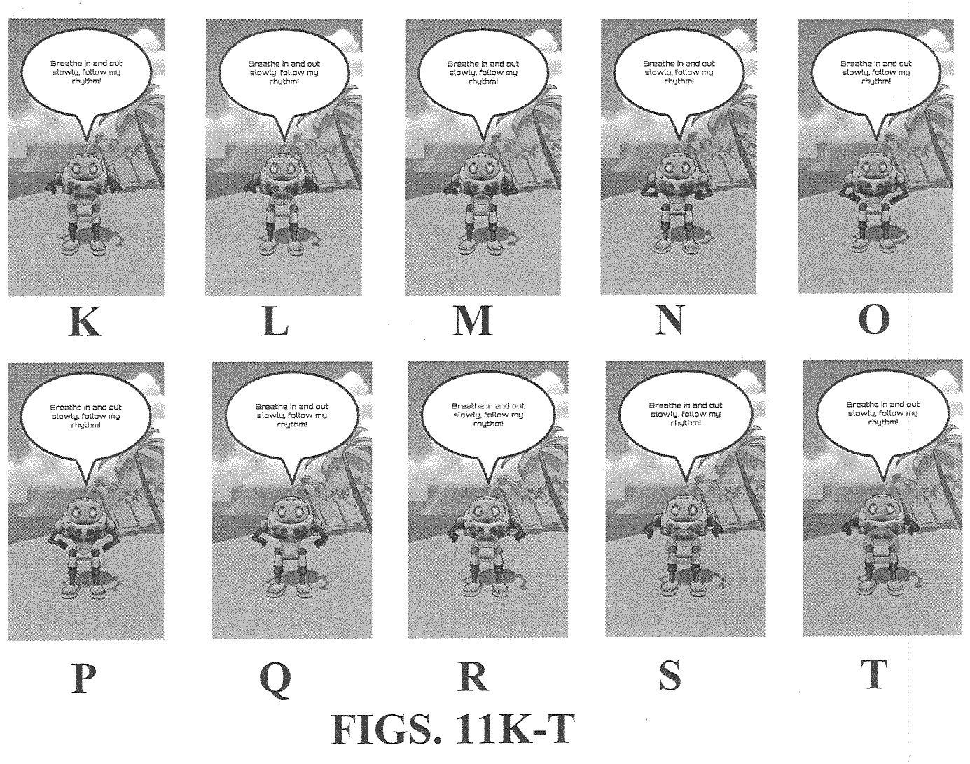

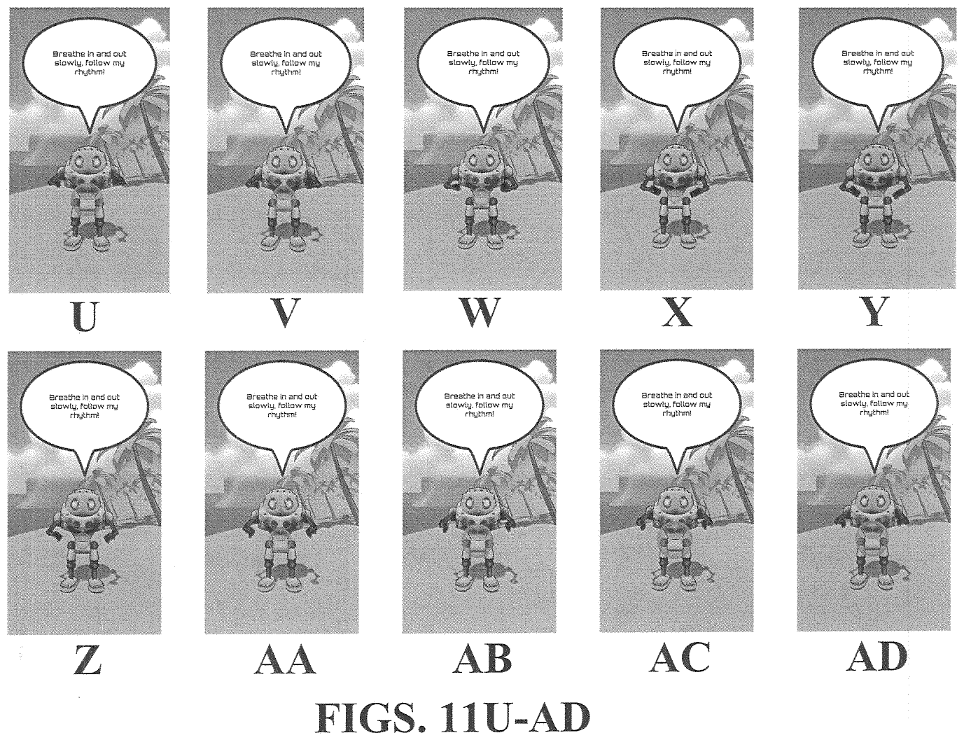

FIG. 10A depicts an inhalation and exhalation simulation including stepwise audio recordings or simulations and a stepwise moonset visualization that grows in intensity during inhalation and fades in intensity during exhalation.

FIG. 10B depicts an inhalation and exhalation simulation including continuous audio recordings or simulations and a stepwise the moonset visualization that grows in intensity during inhalation and fades in intensity during exhalation.

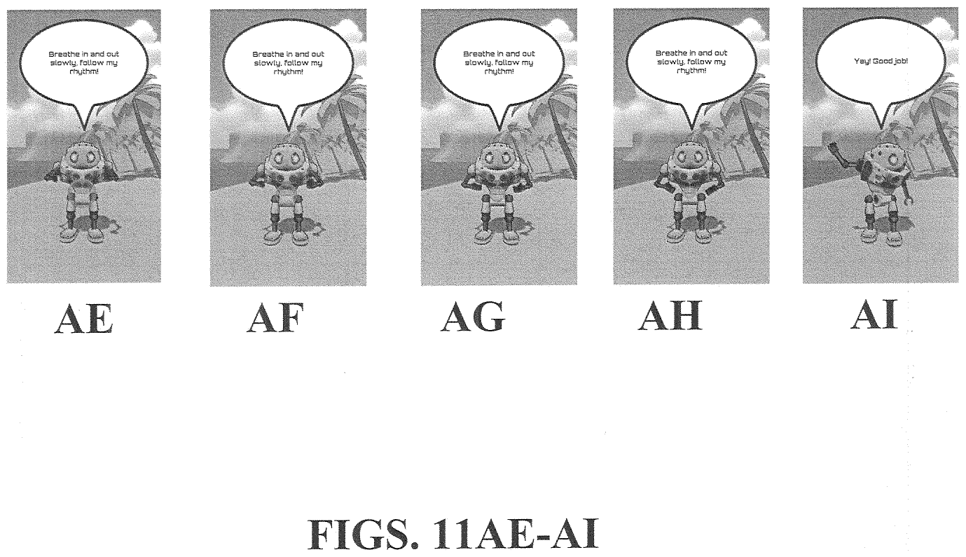

FIGS. 11A-AI depict1 a sequence of simulated avatar images that show activating the avatar and displaying the avatar using a translucent ball to simulate breathing (ball gets bigger during inhalation and smaller during exhalation) and text box displaying text messages to the user as breathing resumes to normal and the information in the text boxes may be heard acoustically or haptically through pulse technology.

It should be recognized that the audio output, the visual output, or the audiovisual output may be derived from natural sounds and/or visuals, simulated sounds and/or visuals, and/or computer generated sounds and/or visuals. It should also be recognized that the audio output, the visual output, the audiovisual output may be change to better assist a user to reestablish a normal breathing pattern or rhythm or to establish a relaxing breathing pattern or rhythm so that the audio output, the visual output, or the audiovisual output may change over time as the user's breathing pattern or rhythm begins to match the audio output, the visual output, or the audiovisual output. It should also be recognized that the audio output, the visual output, or the audiovisual output may be accompanied by an avatar to further assist the user by providing audio and/or visual encouraging comments or visualizations. It should also be recognized that the avatar may be a natural occurring animal or human or the person himself or herself, a simulated naturally occurring animal or human, and/or a computer generated animal or human or other computer generated thing.

### EMBODIMENTS OF THE DISCLOSURE

Embodiment 1. A breathing apparatus comprising:

one or more processing units, each of the processing units including an operating system, a memory, communications hardware and software, one or more mass storage devices, one or more input devices and one or more output devices,

a power supply coupled to or associated with the apparatus,

the apparatus configured to:
acquire, capture, or receive normal breathing data of a user, the normal breathing data comprising normal breathing audio, visual, audiovisual, and/or haptic data;
generate:
   (a) one or more user normal breathing recordings from the normal breathing audio, visual, audiovisual, and/or haptic data, the one or more recordings comprising the audio, visual, audiovisual, and/or haptic data; or
   (b) one or more simulated normal breathing patterns or rhythms from the normal breathing audio, visual, audiovisual, and/or haptic data, the one or more simulated normal breathing patterns or rhythms comprising the audio, visual, audiovisual, and/or haptic data;
while the user undergoes an adverse breathing event, acquire, capture, or receive adverse breathing data, the adverse breathing data comprising adverse breathing audio, visual, audiovisual, and/or haptic data; and
output:
   (a) a normal breathing recording; or
   (b) a simulated normal breathing pattern or rhythm.

Embodiment 2. The apparatus of Embodiment 1, wherein the apparatus is further configured to:
prior to the acquire, capture, or receive adverse breathing data, continually monitor breathing data of the user;
acquire and/or receive initial breathing data from the user undergoing the adverse breathing event;
select a breathing output from the one or more normal breathing recordings or the one or more simulated normal breathing patterns or rhythms based on:
   input from the user; or
   an automatic selection;
continue the output of the normal breathing recording or the simulated normal breathing pattern or rhythm, until a normal breathing pattern or rhythm is reestablished.

Embodiment 3. The apparatus of any of the preceding Embodiments, wherein the apparatus is further configured to:
after the normal breathing pattern or rhythm is reestablished, monitor the user breathing data.

Embodiment 4. The apparatus of any of the preceding Embodiments, wherein the apparatus is further configured to:
during the adverse breathing event, modify the normal breathing recording or the normal breathing simulated normal breathing pattern or rhythm to improve how rapidly the user normal breathing pattern or rhythm is reestablished.

Embodiment 5. The apparatus of any of the preceding Embodiments, wherein the breathing output is selected to best assist the user in reestablishing a normal breathing pattern or rhythm based on the nature of the adverse breathing event the user is experiencing.

Embodiment 6. The apparatus of any of the preceding Embodiments, wherein the apparatus is further configured to:
while the user undergoes the adverse breathing event, determine differences between the normal user breathing pattern or rhythm and the adverse user breathing pattern or rhythm; and
output the differences along with the normal breathing recording or the a simulated normal breathing pattern or rhythm.

Embodiment 7. The apparatus of any of the preceding Embodiments, wherein the apparatus is further configured to:
while the user undergoes the adverse breathing event, highlight the differences between the normal user breathing pattern or rhythm and the adverse user breathing pattern or rhythm.

Embodiment 8. The apparatus of any of the preceding Embodiments, wherein the apparatus is further configured to:
while the user undergoes the adverse breathing event, change highlight as the differences lessen.

Embodiment 9. A breathing system comprising:
one or more processing units, each of the processing units including an operating system, a memory, communications hardware and software, one or more mass storage devices, one or more input devices and one or more output devices,
a power supply coupled to or associated with the system,
the system configured to:
   acquire, capture, or receive normal breathing data of a user, the normal breathing data comprising normal breathing audio, visual, audiovisual, and/or haptic data;
   generate:
      (a) one or more user normal breathing recordings from the normal breathing audio, visual, audiovisual, and/or haptic data, the one or more recordings comprising the audio, visual, audiovisual, and/or haptic data; or
      (b) one or more simulated normal breathing patterns or rhythms from the normal breathing audio, visual, audiovisual, and/or haptic data, the one or more simulated normal breathing patterns or rhythms comprising the audio, visual, audiovisual, and/or haptic data;
   while the user undergoes an adverse breathing event, acquire, capture, or receive adverse breathing data, the adverse breathing data comprising adverse breathing audio, visual, audiovisual, and/or haptic data; and output:
      (a) a normal breathing recording; or
      (b) a simulated normal breathing pattern or rhythm.

Embodiment 10. The system of Embodiment 1, wherein the system is further configured to:
prior to the acquire, capture, or receive adverse breathing data, continually monitor breathing data of the user;
acquire and/or receive initial breathing data from the user undergoing the adverse breathing event;
select a breathing output from the one or more normal breathing recordings or the one or more simulated normal breathing patterns or rhythms based on:
   input from the user; or
   an automatic selection;
continue the output of the normal breathing recording or the simulated normal breathing pattern or rhythm, until a normal breathing pattern or rhythm is reestablished.

Embodiment 11. The system of any of the preceding Embodiments, wherein the system is further configured to:
after the normal breathing pattern or rhythm is reestablished, monitor the user breathing data.

Embodiment 12. The system of any of the preceding Embodiments, wherein the system is further configured to:
during the adverse breathing event, modify the normal breathing recording or the normal breathing simulated normal breathing pattern or rhythm to improve how rapidly the user normal breathing pattern or rhythm is reestablished.

Embodiment 13. The system of any of the preceding Embodiments, wherein the breathing output is selected to best assist the user in reestablishing a normal breathing pattern or rhythm based on the nature of the adverse breathing event the user is experiencing.

Embodiment 14. The system of any of the preceding Embodiments, wherein the system is further configured to:
while the user undergoes the adverse breathing event, determine differences between the normal user breathing pattern or rhythm and the adverse user breathing pattern or rhythm; and
output the differences along with the normal breathing recording or the a simulated normal breathing pattern or rhythm.

Embodiment 15. The system of any of the preceding Embodiments, wherein the system is further configured to:
while the user undergoes the adverse breathing event, highlight the differences between the normal user breathing pattern or rhythm and the adverse user breathing pattern or rhythm.

Embodiment 16. The system of any of the preceding Embodiments, wherein the system is further configured to:
while the user undergoes the adverse breathing event, change highlight as the differences lessen.

Embodiment 17. A breathing interface comprising:
one or more processing units, each of the processing units including an operating system, a memory, communications hardware and software, one or more mass storage devices, one or more input devices and one or more output devices,
a power supply coupled to or associated with the interface,
the interface configured to:
   acquire, capture, or receive normal breathing data of a user, the normal breathing data comprising normal breathing audio, visual, audiovisual, and/or haptic data;
   generate:
      (a) one or more user normal breathing recordings from the normal breathing audio, visual, audiovisual, and/or haptic data, the one or more recordings comprising the audio, visual, audiovisual, and/or haptic data; or
      (b) one or more simulated normal breathing patterns or rhythms from the normal breathing audio, visual, audiovisual, and/or haptic data, the one or more simulated normal breathing patterns or rhythms comprising the audio, visual, audiovisual, and/or haptic data;
   while the user undergoes an adverse breathing event, acquire, capture, or receive adverse breathing data, the adverse breathing data comprising adverse breathing audio, visual, audiovisual, and/or haptic data; and output:
      (a) a normal breathing recording; or
      (b) a simulated normal breathing pattern or rhythm.

Embodiment 18. The interface of Embodiment 1, wherein the interface is further configured to:
prior to the acquire, capture, or receive adverse breathing data, continually monitor breathing data of the user;
acquire and/or receive initial breathing data from the user undergoing the adverse breathing event;
select a breathing output from the one or more normal breathing recordings or the one or more simulated normal breathing patterns or rhythms based on:
   input from the user; or
   an automatic selection;
continue the output of the normal breathing recording or the simulated normal breathing pattern or rhythm, until a normal breathing pattern or rhythm is reestablished.

Embodiment 19. The interface of any of the preceding Embodiments, wherein the interface is further configured to:
after the normal breathing pattern or rhythm is reestablished, monitor the user breathing data.

Embodiment 20. The interface of any of the preceding Embodiments, wherein the interface is further configured to:
during the adverse breathing event, modify the normal breathing recording or the normal breathing simulated normal breathing pattern or rhythm to improve how rapidly the user normal breathing pattern or rhythm is reestablished.

Embodiment 21. The interface of any of the preceding Embodiments, wherein the breathing output is selected to best assist the user in reestablishing a normal breathing pattern or rhythm based on the nature of the adverse breathing event the user is experiencing.

Embodiment 22. The interface of any of the preceding Embodiments, wherein the interface is further configured to:
while the user undergoes the adverse breathing event, determine differences between the normal user breathing pattern or rhythm and the adverse user breathing pattern or rhythm; and
output the differences along with the normal breathing recording or the a simulated normal breathing pattern or rhythm.

Embodiment 23. The interface of any of the preceding Embodiments, wherein the interface is further configured to:
while the user undergoes the adverse breathing event, highlight the differences between the normal user breathing pattern or rhythm and the adverse user breathing pattern or rhythm.

Embodiment 24. The interface of any of the preceding Embodiments, wherein the interface is further configured to:
while the user undergoes the adverse breathing event, change highlight as the differences lessen.

Embodiment 25. A method, implemented on an apparatus, system, or interface comprising (a) one or more processing units, each of the processing units including an operating system, a memory, communications hardware and software, one or more mass storage devices, one or more input devices and one or more output devices, and (b) a power supply coupled to or associated with the apparatus, system, or interface, the method comprising:
acquiring, capturing, or receiving normal breathing data of a user, the normal breathing data comprising normal breathing audio, visual, audiovisual, and/or haptic data;
   generating:
      (a) one or more user normal breathing recordings from the normal breathing audio, visual, audiovisual, and/or haptic data, the one or more recordings comprising the audio, visual, audiovisual, and/or haptic data; or
      (b) one or more simulated normal breathing patterns or rhythms from the normal breathing audio, visual, audiovisual, and/or haptic data, the one or more simulated normal breathing patterns or rhythms comprising the audio, visual, audiovisual, and/or haptic data;
   while the user undergoes an adverse breathing event, acquiring, capturing, or receiving adverse breathing data, the adverse breathing data comprising adverse breathing audio, visual, audiovisual, and/or haptic data; and
   outputting:
      (a) a normal breathing recording; or
      (b) a simulated normal breathing pattern or rhythm.

Embodiment 26. The method of Embodiment 1, further comprising:
prior to the acquiring, capturing, or receiving adverse breathing data, continue monitoring breathing data of the user;
acquiring, capturing, or receiving initial breathing data from the user undergoing the adverse breathing event;
selecting a breathing output from the one or more normal breathing recordings or the one or more simulated normal breathing patterns or rhythms based on:
   receiving input from the user; or
   an automatic selecting the breathing output;
continuing the outputting of the normal breathing recording or the simulated normal breathing pattern or rhythm, until a normal breathing pattern or rhythm is reestablished.

Embodiment 27. The method of any of the preceding Embodiments, further comprising:
after the normal breathing pattern or rhythm is reestablished, continue monitoring the user breathing data.

Embodiment 28. The method of any of the preceding Embodiments, further comprising:
during the adverse breathing event, modifying the normal breathing recording or the normal breathing simulated normal breathing pattern or rhythm to improve how rapidly the user normal breathing pattern or rhythm is reestablished.

Embodiment 29. The method of any of the preceding Embodiments, wherein, in the outputting step, the breathing output is selected to best assist the user in reestablishing a normal breathing pattern or rhythm based on the nature of the adverse breathing event the user is experiencing.

Embodiment 30. The method of any of the preceding Embodiments, further comprising:
while the user undergoes the adverse breathing event, determining differences between the normal user breathing pattern or rhythm and the adverse user breathing pattern or rhythm; and
outputting the differences along with the normal breathing recording or the a simulated normal breathing pattern or rhythm.

Embodiment 31. The method of any of the preceding Embodiments, further comprising:
while the user undergoes the adverse breathing event, highlighting the differences between the normal user breathing pattern or rhythm and the adverse user breathing pattern or rhythm.

Embodiment 32. The method of any of the preceding Embodiments, further comprising:
while the user undergoes the adverse breathing event, changing the highlighting as the differences lessen.

### CLOSING PARAGRAPH OF THE DISCLOSURE

All references cited herein are incorporated by reference. Although the disclosure has been disclosed with reference to its preferred embodiments, from reading this description those of skill in the art may appreciate changes and modification that may be made which do not depart from the scope and spirit of the disclosure as described above and claimed hereafter.

We claim:

1. A breathing apparatus comprising:
one or more processing units, each of the processing units including an operating system, a memory, communications hardware and software, one or more mass storage devices, one or more input devices and one or more output devices,
a power supply coupled to or associated with the apparatus,
the apparatus configured to:
acquire, capture, or receive normal breathing data of a user, the normal breathing data comprising normal breathing audio, visual, audiovisual, and/or haptic data;
generate:
(a) one or more user normal breathing recordings from the normal breathing audio, visual, audiovisual, and/or haptic data, the one or more recordings comprising the audio, visual, audiovisual, and/or haptic data; or
(b) one or more simulated normal breathing patterns or rhythms from the normal breathing audio, visual, audiovisual, and/or haptic data, the one or more simulated normal breathing patterns or rhythms comprising the audio, visual, audiovisual, and/or haptic data;
while the user undergoes an adverse breathing event, acquire, capture, or receive adverse breathing data, the adverse breathing data comprising adverse breathing audio, visual, audiovisual, and/or haptic data; and
output:
(a) a normal breathing recording; or
(b) a simulated normal breathing pattern or rhythm.

2. The apparatus claim 1, further configured to: wherein the apparatus is further configured to:
prior to the acquire, capture, or receive adverse breathing data, continually monitor breathing data of the user;
acquire and/or receive initial breathing data from the user undergoing the adverse breathing event;
select a breathing output from the one or more normal breathing recordings or the one or more simulated normal breathing patterns or rhythms based on:
input from the user; or
an automatic selection;
continue the output of the normal breathing recording or the simulated normal breathing pattern or rhythm, until a normal breathing pattern or rhythm is reestablished.

3. The apparatus claim 1, wherein the apparatus is further configured to:
after the normal breathing pattern or rhythm is reestablished, monitor the user breathing data.

4. The apparatus claim 1, wherein the apparatus is further configured to:
during the adverse breathing event, modify the normal breathing recording or the normal breathing simulated normal breathing pattern or rhythm to improve how rapidly the user normal breathing pattern or rhythm is reestablished.

5. The apparatus claim 1, wherein the breathing output is selected to best assist the user in reestablishing a normal breathing pattern or rhythm based on the nature of the adverse breathing event the user is experiencing.

6. The apparatus claim 1, wherein the apparatus is further configured to:
while the user undergoes the adverse breathing event, determine differences between the normal user breathing pattern or rhythm and the adverse user breathing pattern or rhythm; and
output the differences along with the normal breathing recording or the a simulated normal breathing pattern or rhythm.

7. The apparatus claim 6, wherein the apparatus is further configured to:
while the user undergoes the adverse breathing event, highlight the differences between the normal user breathing pattern or rhythm and the adverse user breathing pattern or rhythm.

8. The apparatus claim 7, wherein the apparatus is further configured to:
while the user undergoes the adverse breathing event, change highlight as the differences lessen.

9. A method, implemented on an apparatus, system, or interface comprising (a) one or more processing units, each of the processing units including an operating system, a memory, communications hardware and software, one or more mass storage devices, one or more input devices and one or more output devices, and (b) a power supply coupled to or associated with the apparatus, system, or interface, the method comprising:
acquiring, capturing, or receiving normal breathing data of a user, the normal breathing data comprising normal breathing audio, visual, audiovisual, and/or haptic data;
generating:
(a) one or more user normal breathing recordings from the normal breathing audio, visual, audiovisual, and/or haptic data, the one or more recordings comprising the audio, visual, audiovisual, and/or haptic data; or
(b) one or more simulated normal breathing patterns or rhythms from the normal breathing audio, visual, audiovisual, and/or haptic data, the one or more simulated normal breathing patterns or rhythms comprising the audio, visual, audiovisual, and/or haptic data;
while the user undergoes an adverse breathing event, acquiring, capturing, or receiving adverse breathing data, the adverse breathing data comprising adverse breathing audio, visual, audiovisual, and/or haptic data; and
outputting:
(a) a normal breathing recording; or
(b) a simulated normal breathing pattern or rhythm.

10. The method of claim 9, further comprising:
further comprising:
prior to the acquiring, capturing, or receiving adverse breathing data, continue monitoring breathing data of the user;
acquiring, capturing, or receiving initial breathing data from the user undergoing the adverse breathing event;
selecting a breathing output from the one or more normal breathing recordings or the one or more simulated normal breathing patterns or rhythms based on:
receiving input from the user; or
an automatic selecting the breathing output;
continuing the outputting of the normal breathing recording or the simulated normal breathing pattern or rhythm, until a normal breathing pattern or rhythm is reestablished.

11. The method of claim 9, further comprising:
after the normal breathing pattern or rhythm is reestablished, continue monitoring the user breathing data.

12. The method of claim 9, further comprising:
during the adverse breathing event, modifying the normal breathing recording or the normal breathing simulated normal breathing pattern or rhythm to improve how rapidly the user normal breathing pattern or rhythm is reestablished.

13. The method of claim 9, wherein, in the outputting step, the breathing output is selected to best assist the user in reestablishing a normal breathing pattern or rhythm based on the nature of the adverse breathing event the user is experiencing.

14. The method of claim 9, further comprising:
while the user undergoes the adverse breathing event, determining differences between the normal user breathing pattern or rhythm and the adverse user breathing pattern or rhythm; and
outputting the differences along with the normal breathing recording or the a simulated normal breathing pattern or rhythm.

15. The method of claim 14, further comprising:
while the user undergoes the adverse breathing event, highlighting the differences between the normal user breathing pattern or rhythm and the adverse user breathing pattern or rhythm.

16. The method of claim 15, further comprising:
while the user undergoes the adverse breathing event, changing the highlighting as the differences lessen.
